(19) United States
(12) Patent Application Publication
(10) Pub. No.: US 2024/0182565 A1
(43) Pub. Date: Jun. 6, 2024

(54) CD33 SPECIFIC CHIMERIC ANTIGEN RECEPTORS FOR CANCER IMMUNOTHERAPY

(71) Applicant: Cellectis, Paris (FR)

(72) Inventor: Roman Galetto, Paris (FR)

(21) Appl. No.: 18/495,109

(22) Filed: Oct. 26, 2023

Related U.S. Application Data
(63) Continuation of application No. 15/926,755, filed on Mar. 20, 2018, now abandoned.
Application No. 15/926,755 is a division of application No. 15/301,686, filed on Oct. 3, 2016, now Pat. No. 9,944,702.

(30) Foreign Application Priority Data
Apr. 3, 2014 (DK) PA201470171

(51) Int. Cl. C07K 16/28; A61K 39/00; C07K 14/705; C07K 14/725

(52) U.S. Cl. CPC C07K 16/2803; C07K 14/7051; C07K 14/70517; A61K 2039/505; C07K 2317/24; C07K 2317/53; C07K 2317/62; C07K 2317/622; C07K 2317/73; C07K 2319/03

(57) ABSTRACT

The present invention relates to Chimeric Antigen Receptors (CAR) that are recombinant chimeric proteins able to redirect immune cell specificity and reactivity toward selected membrane antigens, and more particularly in which extracellular ligand binding is a scFV derived from a CD33 monoclonal antibody, conferring specific immunity against CD33 positive cells. The engineered immune cells endowed with such CARs are particularly suited for treating lymphomas and leukemia.

### SEQUENCE LISTING STATEMENT

The content of the electronic sequence listing titled CELLT-36654-303.xml (Size: 159,990 bytes; and Date of Creation: Feb. 20, 2024) is herein incorporated by reference in its entirety.

### FIELD OF THE INVENTION

The present invention relates to Chimeric Antigen Receptors (CAR) that are recombinant chimeric proteins able to redirect immune cell specificity and reactivity toward CD33, a cell surface glycoprotein found on most myeloid cells and used to diagnose acute myeloid Leukemia (AML) in patients. The CARs according to the invention are particularly useful to treat malignant cells bearing CD33, when expressed in T-cells or NK cells. The resulting engineered immune cells display high level of specificity toward malignant cells, conferring safety and efficiency for immunotherapy.

### BACKGROUND OF THE INVENTION

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T-cell cytotoxicity. However, they failed to provide prolonged expansion and anti-tumor activity in vivo. Signaling domains from co-stimulatory molecules, as well as transmembrane and hinge domains have been added to form CARs of second and third generations, leading to some successful therapeutic trials in humans, where T-cells could be redirected against malignant cells expressing CD19 (June et al., 2011). However, the particular combination of signaling domains, transmembrane and co-stimulatory domains used with respect to CD19 ScFv, was rather antigen-specific and cannot be expanded to any antigen markers.

Acute myeloid leukaemia (AML) is the second most common acute leukaemia with approximately 13,300 new cases per year in the United States and 8,800 annual deaths. The commonly applied therapy of leukaemic diseases includes irradiation and/or chemotherapy. Furthermore, under certain circumstances, the additional possibility of bone marrow transplantation is regarded suitable. However, these therapies are relatively toxic to the patient and very often do not lead to a complete cure from the disease. Thus, although a complete remission can be achieved for 65-80% of patients receiving chemotherapy, most of these patients relapse (Cros et al., 2004) because the cells that survived the chemotherapy are enriched in AML leukaemia stem cells (AML-LSCs), and constitute a particularly dangerous reservoir of cells capable of re-expanding and causing a relapse. Leukaemia stem cells have been particularly well characterized for acute myeloid leukaemia. AML-LSCs express a characteristic set of cell-surface antigens including among others CD33. Patients older than 60 years have a poor prognosis with only 10% to 15% of 4-year disease-free survival (Gardin et al., 2007). This high relapse rate for AML patients and the poor prognosis for older patients highlight the urgent need for novel therapeutics preferentially targeting CD33⁺ cells.

CD33 (Sialic acid-binding Ig-like lectin 3) or SIGLEC3, referred to as P20138 under the UniProtKB/Swiss-Prot protein database, is a transmembrane receptor expressed on cells of myeloid lineage. It is usually considered myeloid-specific, but it can also be found on some lymphoid cells. It binds sialic acids, therefore is a member of the SIGLEC family of lectins.

In the past, different approaches have been used to develop unconjugated monoclonal antibodies with antitumor activity against CD33. However these attempts failed to address malignant cells specifically.

In 2000 Gemtuzumabozogamicin (Mylotarg™, GO), a calicheamicin conjugated humanized anti-CD33 monoclonal antibody, was approved by the American Food and Drug Administration (FDA) for treating patients older than 60 years with refractory or relapsed AML. However this was withdrawn from the market on Jun. 21, 2010; GO consisted of a humanized anti-CD33 IgG-antibody, chemically coupled to the cytotoxic agent calicheamicin. Post-approval study (SWOG S0106) raised concerns about the product's safety, while other clinical trials (British MRC AML-15 and the HOVON-43 trials) failed to demonstrate any clinical benefit (Maniecki et al., 2011). Side effects were found to include hepatic veno-occlusive disease, pulmonary toxicity and severe hypersensitivity reactions, whereas in vitro studies revealed antigen-independent cytotoxicities towards CD33 negative cell lines (Schwemmlein ef al, 2006).

More recently, tri-specific polypeptide molecules combining immunoglobulin domains from CD123, CD16 and CD33 antibodies were proposed (WO2011/070109) to obviate the specificity issues previously encountered with therapeutics targeting CD33.

As an alternative to the previous strategies, the present invention provides with CD33 specific CARs, which can be expressed in immune cells to target CD33+ malignant cells with significant clinical advantage.

### SUMMARY OF THE INVENTION

The inventors have generated CD33 specific CAR having different structure and comprising different scFV derived from different CD33 specific antibodies. Preferred CAR polypeptides of the invention comprise an amino acid sequence selected from SEQ ID NO.19 to 42. More preferred CAR polypeptides of the invention comprise an amino acid sequence of SEQ ID NO. 68, or with at least 80% identity with SEQ ID NO. 68. Following non-specific activation in vitro (e.g. with anti CD3/CD28 coated beads and recombinant IL2), T-cells from donors have been transformed with polynucleotides expressing these CARs using viral transduction. In certain instances, the T-cells were further engineered to create non-alloreactive T-cells, more especially by disruption of a component of TCR (αβ-T-Cell receptors) to prevent Graft versus host reaction and even more especially by disruption of a component of a TCR (αβ-T-Cell receptors) and of a CD33 gene.

The resulting engineered T-cells displayed reactivity in-vitro against CD33 positive cells to various extend, showing that the CARs of the present invention contribute to antigen dependent activation, and also proliferation, of the T-cells, making them useful for immunotherapy.

The polypeptides and polynucleotide sequences encoding the CARs of the present invention are detailed in the present specification.

The engineered immune cells of the present invention are particularly useful for therapeutic applications such as B-cell lymphoma or leukemia treatments.

### BRIEF DESCRIPTION OF THE FIGURES

FIG. 1: Schematic representation of an engineered immune cell according to the invention. The engineered immune cell presented in this figure is a T-cell transduced with a retroviral polypeptide encoding CAR. This T-cell is further engineered to allow a better and safer engraftment into the patient, which is optional within the frame of the present invention. X gene may be for instance a gene expressing a component of TCR (TCRalpha or TCRbeta), Y may be a gene involved into the sensitivity of T-cells to immune-suppressive drugs like CD52 (with respect to Campath) or HPRT (with respect to 6-Thioguanine).

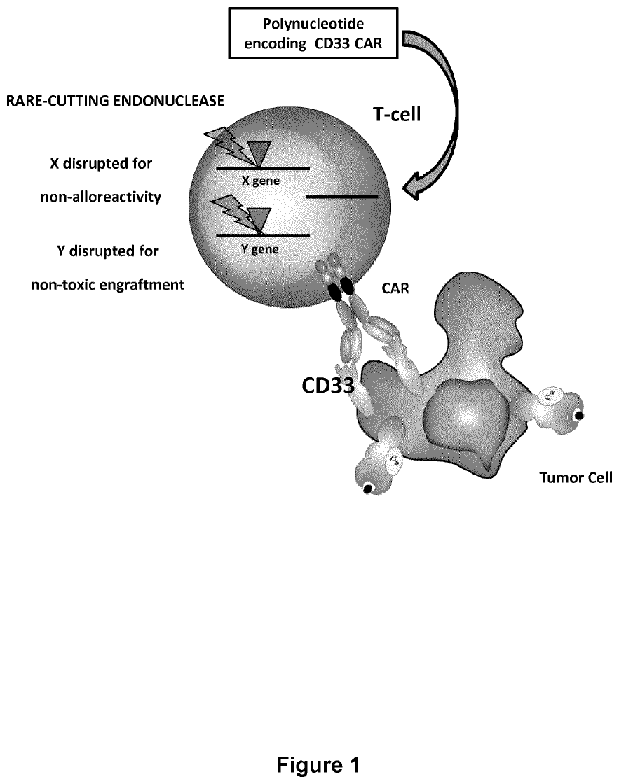

FIG. 2: Schematic representation of the different CAR Architecture (V1 to V6).

FIG. 3: Schematic representation of different CAR with a V1, V3 or V5 structure.

FIG. 4: Degranulation activity (% CD8/CD107a+ cells and mean fluorescence intensity (MFI) CD107a in CD8+ cells) of the M195 scFv with the three architectures (v1: FcgRIII-hinge/CD8-transmembrane, v3: CD8-hinge/CD8-transmembrane, v5: IgG1-hinge/CD8-transmembrane), when CAR+ T-cells were co-cultured for 6 hours with CD33 expressing cells (U937), or with cells that do not express CD33 (Jeko). The data is presented as a percentage (%) of degranulation and mean fluorescence intensity (MFI).

FIG. 5: Degranulation activity (% CD8/CD107a+ cells and MFI CD107a in CD8+ cells) of the m2H12 and My9.6 scFvs with the three architectures (v1: FcgRIII-hinge/CD8-transmembrane, v3: CD8-hinge/CD8-transmembrane, v5: IgG1-hinge/CD8-transmembrane), when CAR+ T-cells were co-cultured for 6 hours with CD33 expressing cells (U937), or with cells that do not express CD33 (Jeko). The data is presented as a percentage (%) of degranulation and mean fluorescence intensity (MFI).

FIG. 6: Degranulation activity (% CD8/CD107a+ cells) of 7 constructions: M195-V1, M195-V3, M195-V5, m2H12-V1, m2H12-V3 and My9.6-V1 and My9.6-V3 (v1: FcgRIII-hinge/CD8-transmembrane, v3: CD8-hinge/CD8-transmembrane, v5: IgG1-hinge/CD8-transmembrane), when CAR+ T-cells were co-cultured for 6 hours with cells expressing high or intermediate levels of CD33 (U937 and K562, respectively), or with cells that do not express CD33 (Jeko-1). The data is presented as a percentage (%) of degranulation.

FIG. 7: Amount of IFN gamma released by anti-CD33 CAR expressing T-cells when co-cultured for 24 h with cells expressing different levels of CD33 (U937 and K562), or with cells that do not express CD33 (Jeko-1). IFN gamma release from T-cells cultured alone, in the same conditions that the co-cultures, is also shown. The experiments were done using three independent donors, and results from one representative donor are shown here.

FIG. 8: Specific cytolytic activity of anti-CD33 CAR expressing-T cells. Assays were done 48 h after CAR mRNA transfection. T-cells were co-cultured with U937+Jeko or K562+Jeko cells for 4 hours. Cellular viability for each of the cell lines was determined at the end of the co-cultured and a specific cell lysis percentage was calculated.

TABLE 1
Sequence of the different CAR components

| Functional domains | SEQ ID # | Raw amino acid sequence |
|---|---|---|
| CD8α signal peptide | SEQ ID NO. 1 | MALPVTALLLPLALLLHAARP |
| Alternative signal peptide | SEQ ID NO. 2 | METDTLLLWVLLLWVPGSTG |
| FcγRIIIα hinge | SEQ ID NO. 3 | GLAVSTISSFFPPGYQ |
| CD8α hinge | SEQ ID NO. 4 | TTTPAPRPPTPAPTIASQPLSLRPEACRPAAGGA |
| | | VHTRGLDFACD |
| IgG1 hinge | SEQ ID NO. 5 | EPKSPDKTHTCPPCPAPPVAGPSVFLFPPKPKDT |
| | | LMIARTPEVTCVVVDVSHEDPEVKFNWYVDGV |
| | | EVHNAKTKPREEQYNSTYRVVSVLTVLHQDWL |
| | | NGKEYKCKVSNKALPAPIEKTISKAKGQPREPQV |
| | | YTLPPSRDELTKNQVSLTCLVKGFYPSDIAVEWE |
| | | SNGQPENNYKTTPPVLDSDGSFFLYSKLTVDKSR |
| | | WQQGNVFSCSVMHEALHNHYTQKSLSLSPGK |
| CD8α transmembrane domain | SEQ ID NO. 6 | IYIWAPLAGTCGVLLLSLVITLYC |
| 41BB transmembrane domain | SEQ ID NO. 7 | IISFFLALTSTALLFLLFFLTLRFSVV |
| 41BB intracellular domain | SEQ ID NO. 8 | KRGRKKLLYIFKQPFMRPVQTTQEEDGCSCRFPE |
| | | EEEGGCEL |
| CD3ζ intracellular domain | SEQ ID NO. 9 | RVKFSRSADAPAYQQGQNQLYNELNLGRREEY |
| | | DVLDKRRGRDPEMGGKPRRKNPQEGLYNELQK |
| | | DKMAEAYSEIGMKGERRRGKGHDGLYQGLSTA |
| | | TKDTYDALHMQALPPR |
| Linker | SEQ ID NO. 10 | GGGGSGGGGSGGGGS |

TABLE 2
Sequence of the different CAR components

| ScFv sequences | SEQ ID # | Raw amino acid sequence |
|---|---|---|
| M195 heavy chain | SEQ ID NO. 11 | EVQLQQSGPELVKPGASVKISCKASGYTFTDYN |
| variable region | | MHWVKQSHGKSLEWIGYIYPYNGGTGYNQKF |
| | | KSKATLTVDNSSSTAYMDVRSLTSEDSAVYYCA |
| | | RGRPAMDYWGQGTSVTVS |
| M195 light chain | SEQ ID NO. 12 | DIVLTQSPASLAVSLGQRATISCRASESVDNYGI |
| variable region | | SFMNWFQQKPGQPPKLLIYAASNQGSGVPAR |
| | | FSGSGSGTDFSLNIHPMEEDDTAMYFCQQSKE |
| | | VPWTFGGGTKLEIK |
| M2H12 heavy chain | SEQ ID NO. 13 | QVQLQQSGPELVRPGTFVKISCKASGYTFTNYD |
| variable region | | INWVNQRPGQGLEWIGWIYPGDGSTKYNEKF |
| | | KAKATLTADKSSSTAYLQLNNLTSENSAVYFCAS |
| | | GYEDAMDYWGQGTSVTVSS |
| M2H12 light chain | SEQ ID NO. 14 | DIKMTQSPSSMYASLGERVIINCKASQDINSYLS |
| variable region | | WFQQKPGKSPKTLIYRANRLVDGVPSRFSGSG |
| | | SGQDYSLTISSLEYEDMGIYYCLQYDEFPLTFGA |
| | | GTKLELKR |
| DRB2heavy chain | SEQ ID NO. 15 | EVKLQESGPELVKPGASVKMSCKASGYKFTDYV |
| variable region | | VHWLKQKPGQGLEWIGYINPYNDGTKYNEKF |
| | | KGKATLTSDKSSSTAYMEVSSLTSEDSAVYYCA |
| | | RDYRYEVYGMDYWGQGTSVTVSS |
| DRB2 light chain | SEQ ID NO. 16 | DIVLTQSPTIMSASPGERVTMTCTASSSVNYIH |
| variable region | | WYQQKSGDSPLRWIFDTSKVASGVPARFSGSG |
| | | SGTSYSLTISTMEAEDAATYYCQQWRSYPLTFG |
| | | DGTRLELKRADAAPTVS |
| My9-6 heavy chain | SEQ ID NO. 17 | QVQLQQPGAEVVKPGASVKMSCKASGYTFTS |
| variable region | | YYIHWIKQTPGQGLEWVGVIYPGNDDISYNQK |
| | | FKGKATLTADKSSTTAYMQLSSLTSEDSAVYYC |
| | | AREVRLRYFDVWGAGTTVTVSS |
| My9-6 light chain | SEQ ID NO. 18 | NIMLTQSPSSLAVSAGEKVTMSCKSSQSVFFSS |
| variable region | | SQKNYLAWYQQIPGQSPKWYWASTRESGVP |
| | | DRFTGSGSGTDFTLTISSVQSEDLAIYYCHQYLS |
| | | SRTFGGGTKLEIKR |

TABLE 3
CAR of structure V-1

| | CAR Structure | | | | | | |
|---|---|---|---|---|---|---|---|
| CAR | signal | | | | | | |
| Designation | peptide | | | | | | |
| V-1 | (optional) | VH | VL | FcγRIIIα hinge | CD8α TM | 41BB -IC | CD3ζ CD |
| M195-1 | SEQ ID | SEQ ID | SEQ ID | SEQ ID | SEQ ID | SEQ ID | SEQ ID |
| (SEQ ID NO. 19) | NO. 1 | NO. 11 | NO. 12 | NO. 3 | NO. 6 | NO. 8 | NO. 9 |
| M2H12-1 | SEQ ID | SEQ ID | SEQ ID | SEQ ID | SEQ ID | SEQ ID | SEQ ID |
| (SEQ ID NO. 20) | NO. 1 | NO. 13 | NO. 14 | NO. 3 | NO. 6 | NO. 8 | NO. 9 |
| DRB2-1 | SEQ ID | SEQ ID | SEQ ID | SEQ ID | SEQ ID | SEQ ID | SEQ ID |
| (SEQ ID NO. 21) | NO. 1 | NO. 15 | NO. 16 | NO. 3 | NO. 6 | NO. 8 | NO. 9 |
| My9-6-1 | SEQ ID | SEQ ID | SEQ ID | SEQ ID | SEQ ID | SEQ ID | SEQ ID |
| (SEQ ID NO. 22) | NO. 1 | NO. 17 | NO. 18 | NO. 3 | NO. 6 | NO. 8 | NO. 9 |

TABLE 4
CAR of structure V-2

| | CAR Structure | | | | | | |
|---|---|---|---|---|---|---|---|
| CAR | signal | | | | | | |
| Designation | peptide | | | | | | |
| V-2 | (optional) | VH | VL | FcγRIIIα hinge | 41BB-TM | 41BB -IC | CD3ζ CD |
| M195-2 | SEQ ID | SEQ ID | SEQ ID | SEQ ID | SEQ ID | SEQ ID | SEQ ID |
| (SEQ ID NO. 23) | NO. 1 | NO. 11 | NO. 12 | NO. 3 | NO. 7 | NO. 8 | NO. 9 |
| M2H12-2 | SEQ ID | SEQ ID | SEQ ID | SEQ ID | SEQ ID | SEQ ID | SEQ ID |
| (SEQ ID NO. 24) | NO. 1 | NO. 13 | NO. 14 | NO. 3 | NO. 7 | NO. 8 | NO. 9 |
| DRB2-2 | SEQ ID | SEQ ID | SEQ ID | SEQ ID | SEQ ID | SEQ ID | SEQ ID |
| (SEQ ID NO. 25) | NO. 1 | NO. 15 | NO. 16 | NO. 3 | NO. 7 | NO. 8 | NO. 9 |
| My9-6-2 | SEQ ID | SEQ ID | SEQ ID | SEQ ID | SEQ ID | SEQ ID | SEQ ID |
| (SEQ ID NO. 26) | NO. 1 | NO. 17 | NO. 18 | NO. 3 | NO. 7 | NO. 8 | NO. 9 |

TABLE 5
CAR of structure V-3

| | CAR Structure | | | | | | |
|---|---|---|---|---|---|---|---|
| CAR | signal | | | | | | |
| Designation | peptide | | | | | | |
| V-3 | (optional) | VH | VL | CD8 [symbol] hinge | CD8 [symbol] TM | 41BB -IC | CD3 [symbol] [symbol] CD |
| M195-3 | SEQ ID | SEQ ID | SEQ ID | SEQ ID | SEQ ID | SEQ ID | SEQ ID |
| (SEQ ID NO. 27) | NO. 1 | NO. 11 | NO. 12 | NO. 4 | NO. 6 | NO. 8 | NO. 9 |
| M2H12-3 | SEQ ID | SEQ ID | SEQ ID | SEQ ID | SEQ ID | SEQ ID | SEQ ID |
| (SEQ ID NO. 28) | NO. 1 | NO. 13 | NO. 14 | NO. 4 | NO. 6 | NO. 8 | NO. 9 |
| DRB2-3 | SEQ ID | SEQ ID | SEQ ID | SEQ ID | SEQ ID | SEQ ID | SEQ ID |
| (SEQ ID NO. 29) | NO. 1 | NO. 15 | NO. 16 | NO. 4 | NO. 6 | NO. 8 | NO. 9 |
| My9-6-3 | SEQ ID | SEQ ID | SEQ ID | SEQ ID | SEQ ID | SEQ ID | SEQ ID |
| (SEQ ID NO. 30) | NO. 1 | NO. 17 | NO. 18 | NO. 4 | NO. 6 | NO. 8 | NO. 9 |

TABLE 6
CAR of structure V-4

| | CAR Structure | | | | | | |
|---|---|---|---|---|---|---|---|
| CAR | signal | | | | | | |
| Designation | peptide | | | | | | |
| V-4 | (optional) | VH | VL | CD8α hinge | 41BB-TM | 41BB -IC | CD3ζ CD |
| M195-4 | SEQ ID | SEQ ID | SEQ ID | SEQ ID | SEQ ID | SEQ ID | SEQ ID |
| (SEQ ID NO. 31) | NO. 1 | NO. 11 | NO. 12 | NO. 4 | NO. 7 | NO. 8 | NO. 9 |
| M2H12-4 | SEQ ID | SEQ ID | SEQ ID | SEQ ID | SEQ ID | SEQ ID | SEQ ID |
| (SEQ ID NO. 32) | NO. 1 | NO. 13 | NO. 14 | NO. 4 | NO. 7 | NO. 8 | NO. 9 |
| DRB2-4 | SEQ ID | SEQ ID | SEQ ID | SEQ ID | SEQ ID | SEQ ID | SEQ ID |
| (SEQ ID NO. 33) | NO. 1 | NO. 15 | NO. 16 | NO. 4 | NO. 7 | NO. 8 | NO. 9 |
| My9-6-5 | SEQ ID | SEQ ID | SEQ ID | SEQ ID | SEQ ID | SEQ ID | SEQ ID |
| (SEQ ID NO. 34) | NO. 1 | NO. 17 | NO. 18 | NO. 4 | NO. 7 | NO. 8 | NO. 9 |

TABLE 7
CAR of structure V-5

| | CAR Structure | | | | | | |
|---|---|---|---|---|---|---|---|
| CAR | signal | | | | | | |
| Designation | peptide | | | | | | |
| V-5 | (optional) | VH | VL | IgG1 hinge | CD8 [symbol] TM | 41BB -IC | CD3 [symbol] [symbol] CD |
| M195-5 | SEQ ID | SEQ ID | SEQ ID | SEQ ID | SEQ ID | SEQ ID | SEQ ID |
| (SEQ ID NO. 35) | NO. 1 | NO. 11 | NO. 12 | NO. 5 | NO. 6 | NO. 8 | NO. 9 |
| M2H12-5 | SEQ ID | SEQ ID | SEQ ID | SEQ ID | SEQ ID | SEQ ID | SEQ ID |
| (SEQ ID NO. 36) | NO. 1 | NO. 13 | NO. 14 | NO. 5 | NO. 6 | NO. 8 | NO. 9 |
| DRB2-5 | SEQ ID | SEQ ID | SEQ ID | SEQ ID | SEQ ID | SEQ ID | SEQ ID |
| (SEQ ID NO. 37) | NO. 1 | NO. 15 | NO. 16 | NO. 5 | NO. 6 | NO. 8 | NO. 9 |
| My9-6-5 | SEQ ID | SEQ ID | SEQ ID | SEQ ID | SEQ ID | SEQ ID | SEQ ID |
| (SEQ ID NO. 38) | NO. 1 | NO. 17 | NO. 18 | NO. 5 | NO. 6 | NO. 8 | NO. 9 |

TABLE 8
CAR of structure V-6

| | CAR Structure | | | | | | |
|---|---|---|---|---|---|---|---|
| CAR | signal | | | | | | |
| Designation | peptide | | | | | | |
| V-6 | (optional) | VH | VL | IgG1 hinge | 41BB-TM | 41BB -IC | CD3ζ CD |
| M195-6 | SEQ ID | SEQ ID | SEQ ID | SEQ ID | SEQ ID | SEQ ID | SEQ ID |
| (SEQ ID NO. 39) | NO. 1 | NO. 11 | NO. 12 | NO. 5 | NO. 7 | NO. 8 | NO. 9 |
| M2H12-6 | SEQ ID | SEQ ID | SEQ ID | SEQ ID | SEQ ID | SEQ ID | SEQ ID |
| (SEQ ID NO. 40) | NO. 1 | NO. 13 | NO. 14 | NO. 5 | NO. 7 | NO. 8 | NO. 9 |
| DRB2-6 | SEQ ID | SEQ ID | SEQ ID | SEQ ID | SEQ ID | SEQ ID | SEQ ID |
| (SEQ ID NO. 41) | NO. 1 | NO. 15 | NO. 16 | NO. 5 | NO. 7 | NO. 8 | NO. 9 |
| My9-6-6 | SEQ ID | SEQ ID | SEQ ID | SEQ ID | SEQ ID | SEQ ID | SEQ ID |
| (SEQ ID NO. 42) | NO. 1 | NO. 17 | NO. 18 | NO. 5 | NO. 7 | NO. 8 | NO. 9 |

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

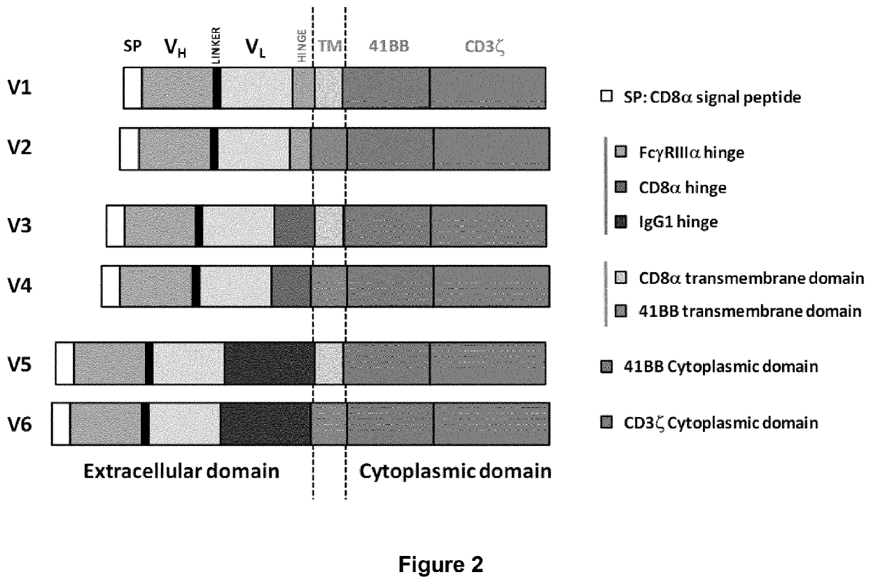

The present invention provides a CD33 specific chimeric antigen receptor (CAR) having at least 80% identity with one of the polypeptide structure selected from V1, V3 and V5 as illustrated in FIG. 2, said structure comprising:
(a) an extra cellular ligand binding-domain comprising VH and VL from a monoclonal anti-CD33 antibody,
(b) a hinge selected from FcRIIIα hinge, CD8α hinge, and IgG1 hinge,
(c) a CD8α transmembrane domain and
(d) a cytoplasmic domain including a CD3 zeta signaling domain and a co-stimulatory domain from 4-1BB.

In a preferred embodiment, the present invention provides a CD33 specific CAR as above wherein said structure V3 comprises a CD8α hinge and a CD8α transmembrane domain.

The present invention provides a CD33 specific CAR as above, wherein said CD8α hinge has at least 80% identity with SEQ ID NO.4.

The present invention provides a CD33 specific CAR as above, which comprises a polypeptide sequence having at least 80% identity with SEQ ID NO. 27, SEQ ID NO.28, SEQ ID NO.29 and SEQ ID NO.30.

In an embodiment, the present invention provides a CD33 specific CAR as above wherein said structure V1 comprises a FcγRIIIα hinge and a CD8α transmembrane domain.

The present invention provides a CD33 specific CAR as above, wherein said FcγRIIIα hinge has at least 80% identity with SEQ ID NO.3.

The present invention provides a CD33 specific CAR as above, which comprises a polypeptide sequence having at least 80% identity with SEQ ID NO. 19, SEQ ID NO.20, SEQ ID NO.21 and SEQ ID NO.22.

In an embodiment, the present invention provides a CD33 specific CAR as above, wherein said structure V5 comprises an IgG1 hinge and a CD8α transmembrane domain.

The present invention provides a CD33 specific CAR as above, wherein said IgG1 hinge has at least 80% identity with SEQ ID NO.5.

The present invention provides a CD33 specific CAR as above, which comprises a polypeptide sequence having at least 80% identity with SEQ ID NO. 35, SEQ ID NO.36, SEQ ID NO.37 and SEQ ID NO.38.

The present invention provides a CD33 specific CAR as above, wherein said VH has at least 80% identity with a polypeptide sequence selected from SEQ ID NO. 11, SEQ ID NO. 13, SEQ ID NO. 15 and SEQ ID NO. 17 and said VL has at least 80% identity with a polypeptide sequence selected from SEQ ID NO. 12, SEQ ID NO. 14, SEQ ID NO. 16 and SEQ ID NO. 18.

The present invention provides a CD33 specific CAR as above, wherein co-stimulatory domain from 4-1BB has at least 80% identity with SEQ ID NO.8.

The present invention provides a CD33 specific CAR as above, wherein said CD3 zeta signaling domain has at least 80% identity with SEQ ID NO. 9.

The present invention provides a CD33 specific CAR as above, wherein said CD8α transmembrane domain has at least 80% identity with SEQ ID NO.6.

The present invention provides a CD33 specific CAR as above, further comprising a signal peptide.

The present invention provides a CD33 specific CAR as above, wherein said signal peptide has at least 80% sequence identity with SEQ ID NO.1 or SEQ ID NO.2.

In one embodiment the present invention provides a CD33 specific CAR as above, which comprises a polypeptide sequence having at least 80% identity with SEQ ID NO. 48 to 71 SEQ ID NO. 48, preferably at least 80% identity with SEQ ID NO. 48, SEQ ID NO. 50, SEQ ID NO. 52, SEQ ID NO. 54, SEQ ID NO. 56, SEQ ID NO. 58, SEQ ID NO. 66, SEQ ID NO. 68, SEQ ID NO. 70, more preferably at least 80% identity with SEQ ID NO. 66, SEQ ID NO. 68, SEQ ID NO. 70.

The present invention provides a CD33 specific CAR as above further comprising another extracellular ligand binding domain which is not specific for CD33.

In one aspect the present invention provides a polynucleotide encoding a CAR according to any one of the embodiments above.

In one aspect the present invention provides an expression vector comprising a polynucleotide as above.

In one aspect, the present invention provides an engineered immune cell expressing at the cell surface membrane a CD33 specific CAR according to any one of the above.

The present invention provides an engineered immune cell as above, derived from inflammatory T-lymphocytes, cytotoxic T-lymphocytes, regulatory T-lymphocytes or helper T-lymphocytes.

The present invention provides an engineered immune cell as above, wherein expression of TCR is suppressed.

The present invention provides an engineered immune cell as above, wherein expression of CD33 is suppressed.

The present invention provides an engineered immune cell as above, wherein said engineered immune cell is modified to be resistant to at least one immune suppressive or chemotherapy drug.

The present invention provides an engineered immune cell as above, for use in therapy.

The present invention provides an engineered immune cell for use in therapy as above, wherein the patient is a human.

The present invention provides an engineered immune cell for use in therapy as above, wherein the condition is a pre-malignant or malignant cancer condition characterized by CD33-expressing cells.

The present invention provides an engineered immune cell for use in therapy as above, wherein the condition is a condition which is characterized by an overabundance of CD33-expressing cells.

The present invention provides an engineered immune cell for use in therapy as above wherein the condition is a hematological cancer condition.

The present invention provides an engineered immune cell for use in therapy as above, wherein the haematological cancer condition is leukemia.

The present invention provides an engineered immune cell for use in therapy as above, wherein said leukemia is selected from the group consisting of acute myelogenous leukemia (AML), chronic myelogenous leukemia, melodysplastic syndrome, acute lymphoid leukemia, chronic lymphoid leukemia, and myelodysplastic syndrome.

The present invention provides an engineered immune cell for use in therapy as above, wherein said leukemia is acute myelogenous leukemia (AML).

The present invention provides an engineered immune cell for use in therapy as above, wherein said hematological cancer is a malignant lymphoproliferative disorder.

The present invention provides an engineered immune cell for use in therapy as above, wherein said malignant lymphoproliferative disorder is a lymphoma.

The present invention provides an engineered immune cell for use in therapy as above, wherein said lymphoma is selected from the group consisting of multiple myeloma, non-Hodgkin's lymphoma, Burkitt's lymphoma, and follicular lymphoma (small cell and large cell).

In one aspect, the present invention provides a method of impairing a hematologic cancer cell comprising contacting said hematologic cancer cell with an engineered immune cell as above in an amount effective to cause impairment of said cancer cell.

In one aspect, the present invention provides a method of engineering an immune cell comprising:
(a) Providing an immune cell,
(b) Expressing at the surface of said cell at least one CD33 specific chimeric antigen receptor as described above.

The present invention provides a method of engineering an immune cell as above comprising:
(a) Providing an immune cell,
(b) Introducing into said cell at least one polynucleotide encoding said CD33 specific chimeric antigen receptor, as above
(c) Expressing said polynucleotide into said cell, optionally expressing said CD33 specific chimeric antigen receptor.
   The present invention provides a method of engineering an immune cell as above further comprising:
(d) Inhibiting TRC expression and/or CD33 expression.

In one embodiment, the immune cell provided for the step (a) of the method described above is an immune cell wherein the expression of TRC and/or CD33 at the cell surface is inhibited, and optionally resistant to at least one drug used to treat a cancer, in particular AML.
The present invention provides a method of engineering an immune cell as above further comprising:
   (a) Introducing at least one other chimeric antigen receptor which is not specific for CD33.
The present invention also provides a method of treating a subject in need thereof comprising:
   (a) Providing an immune cell expressing at the surface a CD33 specific Chimeric Antigen Receptor according to any one of the above;
   (b) Administrating said immune cells to said patient.
In one aspect the present invention provides a method as above, wherein said immune cell is provided from a donor.
The present invention provides a method of treating a subject in need thereof as above wherein said immune cell (to be engineered according to the invention) is provided from the patient himself.

### CD33 Specific Chimeric Antigen Receptors

The present invention relates to new designs of anti-CD33 chimeric antigen receptor (CAR or CD33 CAR or CD33 specific CAR or anti-CD33 CAR) comprising an extracellular ligand-binding domain, a transmembrane domain and a signaling transducing domain.

More precisely, the present invention relates to new CD33 specific CAR comprising an extra cellular ligand binding-domain comprising a VH and a VL from a monoclonal anti-CD33 antibody, a hinge selected from FcRIIIα hinge, CD8alpha hinge and IgG1 hinge, a CD8α transmembrane domain and a cytoplasmic domain including a CD3 zeta signaling domain and a co-stimulatory domain from 4-1BB.

The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. In a preferred embodiment, said extracellular ligand-binding domain comprises a single chain antibody fragment (scFv) comprising the light (V_{L}) and the heavy (V_{H}) variable fragment of a target antigen specific monoclonal anti CD-33 antibody joined by a flexible linker. Said V_{L} and V_{H} are preferably selected from the antibodies referred to as M195, m2H12, DRB2 and My9-6 as indicated in Table 2.

In an even more preferred embodiment said V_{L} and V_{H} comprises SEQ ID NO 17 and 18, optionally humanized.

They are preferably linked together by a flexible linker comprising for instance the sequence SEQ ID NO.10. In other words, said CARs preferentially comprise an extracellular ligand-binding domain comprising a polypeptide sequence displaying at least 90%, 95%, 97% or 99% identity with an amino acid sequence selected from the group consisting of SEQ ID NO: 11 to SEQ ID NO: 18.

In one embodiment, said CAR of the invention preferentially comprises an extracellular ligand-biding domain comprising a polypeptide of SEQ ID NO: 11 and SEQ ID NO: 12.

In one embodiment, said CAR of the invention preferentially comprises an extracellular ligand-biding domain comprising a polypeptide of SEQ ID NO: 13 and SEQ ID NO: 14.

In one embodiment, said CAR of the invention preferentially comprises an extracellular ligand-biding domain comprising a polypeptide of SEQ ID NO: 15 and SEQ ID NO: 16.

In one embodiment, said CAR of the invention preferentially comprises an extracellular ligand-biding domain comprising a polypeptide of SEQ ID NO: 17 and SEQ ID NO: 18.

By the term “recombinant antibody” as used herein, is meant an antibody or antibody fragment which is generated using recombinant DNA technology, such as, for example, an antibody or antibody fragment expressed by a bacteriophage, a yeast expression system or a mammalian cell expression system. The term should also be construed to mean an antibody or antibody fragment which has been generated by the synthesis of a DNA molecule encoding the antibody or antibody fragment and which DNA molecule expresses an antibody or antibody fragment protein, or an amino acid sequence specifying the antibody or antibody fragment, wherein the DNA or amino acid sequence has been obtained using recombinant or synthetic DNA or amino acid sequence technology which is available and well known in the art.

As used herein, the term “conservative sequence modifications” or “humanization” is intended to refer to amino acid modifications that do not significantly affect or alter the characteristics of the CAR (as compared to that of a CAR constructed using the original anti-CD33) and/or that do not significantly affect the activity of the CAR containing the modified amino acid sequence and reduce or abolish a human antimouse antibody (HAMA) response. Such conservative modifications include amino acid substitutions, additions and deletions in said antibody fragment in said CAR and/or any of the other parts of said CAR molecule. Modifications can be introduced into an antibody, into an antibody fragment or in any of the other parts of the CAR molecule of the invention by standard techniques known in the art, such as site-directed mutagenesis, PCR-mediated mutagenesis or by employing optimized germline sequences. Accordingly, the present invention provides a (humanized) CD33 CAR, wherein VH has at least 80% identity with SEQ ID NO 11, SEQ ID NO13, SEQ ID NO15, or SEQ ID NO17 and VL has at least 80% identity with SEQ ID NO 12, SEQ ID NO14, SEQ ID NO16, or SEQ ID NO18.

In one embodiment, said CAR of the invention preferentially comprises an extracellular ligand-biding domain comprising a polypeptide sequence having at least 80%, 81%, 82%, 83%, 84%, 85%, 86%, 87%, 88%, 89%, 90%, 91%, 92%, 93%, 94%, 95%, 96%, 97%, 98%, or 99% identity with an amino acid sequence of SEQ ID NO: 11 and SEQ ID NO 12.

In one embodiment, said CAR of the invention preferentially comprises an extracellular ligand-biding domain comprising a polypeptide sequence having at least 80%, 81%, 82%, 83%, 84%, 85%, 86%, 87%, 88%, 89%, 90%, 91%, 92%, 93%, 94%, 95%, 96%, 97%, 98%, or 99% identity with an amino acid sequence of SEQ ID NO 13 and SEQ ID NO 14.

In one embodiment, said CAR of the invention preferentially comprises an extracellular ligand-biding domain comprising a polypeptide sequence having at least 80%, 81%, 82%, 83%, 84%, 85%, 86%, 87%, 88%, 89%, 90%, 91%, 92%, 93%, 94%, 95%, 96%, 97%, 98%, or 99% identity with an amino acid sequence of SEQ ID NO 15 and SEQ ID NO 16.

In one embodiment, said CAR of the invention preferentially comprises an extracellular ligand-biding domain comprising a polypeptide sequence having at least 80%, 81%, 82%, 83%, 84%, 85%, 86%, 87%, 88%, 89%, 90%, 91%, 92%, 93%, 94%, 95%, 96%, 97%, 98%, or 99% identity with an amino acid sequence of SEQ ID NO 17 and SEQ ID NO 18.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested for the ability to bind CD 33 using the functional assays described herein.

The signal transducing domain or intracellular signaling domain of a CAR according to the present invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector signal function signal and directs the cell to perform a specialized function.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the CAR can comprise the CD3zeta signaling domain which has amino acid sequence with at least 70%, preferably at least 80%, more preferably at least 90%, 95% 97%-99% or 100% sequence identity with amino acid sequence selected from the group consisting of (SEQ ID NO: 9).

In particular embodiment the signal transduction domain of the CAR of the present invention comprises a co-stimulatory signal molecule. A co-stimulatory molecule is a cell surface molecule other than an antigen receptor or their ligands that is required for an efficient immune response. “Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In a preferred embodiment, the signal transduction domain of the CAR of the present invention comprises a part of co-stimulatory signal molecule selected from the group consisting of fragment of 4-1BB (GenBank: AAA53133.) and CD28 (NP_006130.1). In particular the signal transduction domain of the CAR of the present invention comprises amino acid sequence which comprises at least 70%, preferably at least 80%, more preferably at least 90%, 95% 97% 699% or 100% sequence identity with amino acid sequence selected from the group consisting of SEQ ID NO: 8.

A CAR according to the present invention is expressed on the surface membrane of the cell. Thus, such CAR further comprises a transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T-cell receptor such as α, β, γ or δ, polypeptide constituting CD3 complex, IL2 receptor p55 (α chain), p75 (β chain) or γ chain, subunit chain of Fc receptors, in particular Fcγ receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1) The transmembrane domain can further comprise a hinge region between said extracellular ligand-binding domain and said transmembrane domain. The term “hinge region” used herein generally means any oligo- or polypeptide that functions to link the transmembrane domain to the extracellular ligand-binding domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively the hinge region may be a synthetic sequence that corresponds to a naturally occurring hinge sequence, or may be an entirely synthetic hinge sequence. In a preferred embodiment said hinge domain comprises a part of human CD8 alpha chain, FcγRIIIα receptor or IgG1 respectively referred to in this specification as SEQ ID NO. 3, SEQ ID NO. 4 and SEQ ID NO.5, or hinge polypeptides which display preferably at least 80%, more preferably at least 90%, 95% 97% 99% or 100% sequence identity with these polypeptides.

A CAR according to the invention generally further comprises a transmembrane domain (TM) more particularly selected from CD8α and 4-1BB, showing identity with the polypeptides of SEQ ID NO. 6 or 7.

A CAR according to the invention generally comprises a transmembrane domain (TM) from CD8α showing 90%, 91%, 92%, 93%, 94%, 95%, 96%, 97%, 98%, 99% or 100% identity with the polypeptide of SEQ ID NO. 6. In a preferred embodiment, a CAR according to the invention generally further comprises a transmembrane domain (TM) from CD8α showing 100% identity with the polypeptide of SEQ ID NO. 6.

Downregulation or mutation of target antigens is commonly observed in cancer cells, creating antigen-loss escape variants. Thus, to offset tumor escape and render immune cell more specific to target, the CD33 specific CAR according to the invention can comprise another extracellular ligand-binding domains, to simultaneously bind different elements in target thereby augmenting immune cell activation and function. In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the CAR. In another embodiment, the present invention relates to a population of CARs comprising each one different extracellular ligand binding domains. In a particular, the present invention relates to a method of engineering immune cells comprising providing an immune cell and expressing at the surface of said cell a population of CAR each one comprising different extracellular ligand binding domains. In another particular embodiment, the present invention relates to a method of engineering an immune cell comprising providing an immune cell and introducing into said cell polynucleotides encoding polypeptides composing a population of CAR each one comprising different extracellular ligand binding domains. By population of CARs, it is meant at least two, three, four, five, six or more CARs each one comprising different extracellular ligand binding domains. The different extracellular ligand binding domains according to the present invention can preferably simultaneously bind different elements in target thereby augmenting immune cell activation and function. The present invention also relates to an isolated immune cell which comprises a population of CARs each one comprising different extracellular ligand binding domains.

The present invention provides a CD33 specific CAR having one of the polypeptide structure selected from V1 to V6 as illustrated in FIG. 2, said structure comprising an extra cellular ligand binding-domain comprising VH and VL from a monoclonal anti-CD33 antibody, a hinge selected from FcRIIIα hinge, CD8α hinge, and IgG1 hinge, a CD8α transmembrane domain and a cytoplasmic domain including a CD3 zeta signaling domain and a co-stimulatory domain from 4-1BB.

The present invention provides a CD33 specific CAR having one of the polypeptide structure selected from V1, V3 and V5 as illustrated in FIG. 2, said structure comprising an extra cellular ligand binding-domain comprising VH and VL from a monoclonal anti-CD33 antibody, a hinge selected from a FcRIIIα hinge and CD8 alpha (a) hinge, a CD8α transmembrane domain and a cytoplasmic domain including a CD3 zeta signaling domain and a co-stimulatory domain from 4-1BB.

The present invention provides a CD33 CAR comprising a polypeptide sequence selected from the group consisting of SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59; SEQ ID NO: 60, SEQ ID NO: 61, SEQ ID NO: 62, SEQ ID NO: 63, SEQ ID NO: 64, SEQ ID NO: 65?SEQ ID NO: 66, SEQ ID NO: 67, SEQ ID NO: 68, SEQ ID NO: 69, SEQ ID NO:70, SEQ ID NO: 71, preferably said CAR comprises a polypeptide sequence selected from SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59; SEQ ID NO: 66, SEQ ID NO: 67, SEQ ID NO: 68, SEQ ID NO: 69, SEQ ID NO:70, SEQ ID NO: 71 and more preferably, said CAR preferentially comprises a polypeptide sequence selected from SEQ ID NO: 66, SEQ ID NO: 67, SEQ ID NO: 68, SEQ ID NO: 69, SEQ ID NO:70, SEQ ID NO: 71, and even more preferably, said CAR preferentially comprises a polypeptide of SEQ ID NO:68.

The present invention also provides:
A CD33 specific chimeric antigen receptor (CAR) comprising a polypeptide sequence having at least 80%, 81%, 82%, 83%, 84%, 85%, 86%, 87%, 88%, 89%, 90%, 91%, 92%, 93%, 94%, 95%, 96%, 97%, 98% or 99% identity with an amino acid sequence selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO:70,
more preferably, a CAR comprising a polypeptide sequence displaying at least 80%, 81%, 82%, 83%, 84%, 85%, 86%, 87%, 88%, 89%, 90%, 91%, 92%, 93%, 94%, 95%, 96%, 97%, 98%, or 99% identity with amino acid sequences consisting of SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, SEQ ID NO:70,
even more preferably, a CARs comprising a polypeptide sequence displaying at least 80%, 81%, 82%, 83%, 84%, 85%, 86%, 87%, 88%, 89%, 90%, 91%, 92%, 93%, 94%, 95%, 96%, 97%, 98%, or 99% identity with amino acid sequences consisting of SEQ ID NO: 66, SEQ ID NO: 68, SEQ ID NO:70,

Preferably, the present invention provides a CD33 specific CAR having a structure V1 as illustrated in FIG. 2, said structure comprising an extra cellular ligand binding-domain comprising VH and VL from a monoclonal anti-CD33 antibody, a FcRIIIα hinge, a CD8α transmembrane domain and a cytoplasmic domain including a CD3 zeta signaling domain and a co-stimulatory domain from 4-1BB.

Preferably, the present invention provides a CD33 specific CAR having one of the polypeptide structure V1 as illustrated in FIG. 2, said structure comprising an extra cellular ligand binding-domain comprising VH and VL from a monoclonal anti-CD33 antibody selected from M195, m2 h12, and My9.6, optionally humanized, a FcRIIIα hinge, a CD8α transmembrane domain and a cytoplasmic domain including a CD3 zeta signaling domain and a co-stimulatory domain from 4-1BB.

More preferably, the present invention provides a CD33 specific chimeric antigen receptor (CAR) having one of the polypeptide structure V1 as illustrated in FIG. 2, comprising an amino acid sequence selected from the group consisting of SEQ ID NO: 48, SEQ ID NO: 54, SEQ ID NO: 60, and SEQ ID NO: 66, more preferably said CAR preferentially comprises a polypeptide sequence comprising and amino acid sequences consisting of SEQ ID NO: 48, SEQ ID NO: 54, and SEQ ID NO: 66.

Preferably, the present invention provides a CD33 specific chimeric antigen receptor (CAR) having a structure V3 as illustrated in FIG. 2, said structure comprising an extra cellular ligand binding-domain comprising VH and VL from a monoclonal anti-CD33 antibody, CD8α hinge, a CD8α transmembrane domain and a cytoplasmic domain including a CD3 zeta signaling domain and a co-stimulatory domain from 4-1BB.

Preferably, the present invention provides a CD33 specific CAR having a structure V3 as illustrated in FIG. 2, said structure comprising an extra cellular ligand binding-domain comprising VH and VL from a monoclonal anti-CD33 antibody selected from M195, m2 h12, DRB2, and My9.6, optionally humanized, a FcRIIIα hinge, a CD8α transmembrane domain and a cytoplasmic domain including a CD3 zeta signaling domain and a co-stimulatory domain from 4-1BB.

More preferably, the present invention provides a CD33 specific CAR having a structure V3 as illustrated in FIG. 2, comprising an amino acid sequence selected from the group consisting of SEQ ID NO: 50, SEQ ID NO: 56, SEQ ID NO: 62, and SEQ ID NO: 68, more preferably said CAR preferentially comprises a polypeptide sequence comprising and amino acid sequences consisting of SEQ ID NO: 50, SEQ ID NO: 56, and SEQ ID NO: 68.

Preferably, the present invention provides a CD33 specific CAR having a structure V5 as illustrated in FIG. 2, said structure comprising an extra cellular ligand binding-domain comprising VH and VL from a monoclonal anti-CD33 antibody, a IgG1 hinge, a CD8α transmembrane domain and a cytoplasmic domain including a CD3 zeta signaling domain and a co-stimulatory domain from 4-1BB.

Preferably, the present invention provides a CD33 specific CAR having a structure V5 as illustrated in FIG. 2, said structure comprising an extra cellular ligand binding-domain comprising VH and VL from a monoclonal anti-CD33 antibody selected from M195, m2 h12, DRB2, and My9.6, optionally humanized, a FcRIIIα hinge, a CD8α transmembrane domain and a cytoplasmic domain including a CD3 zeta signaling domain and a co-stimulatory domain from 4-1BB.

More preferably, the present invention provides a CD33 specific CAR having a structure V5 as illustrated in FIG. 2, comprising an amino acid sequence selected from the group consisting of SEQ ID NO: 52, SEQ ID NO: 58, SEQ ID NO: 64, and SEQ ID NO: 70, more preferably said CAR preferentially comprises a polypeptide sequence comprising and amino acid sequences consisting of SEQ ID NO: 52, SEQ ID NO: 58, and SEQ ID NO: 70.

In one embodiment, the present invention provides a CD33 specific chimeric antigen receptor comprising:
a optional signal peptide having an amino acid sequence with at least 80%, more preferably at least 90%, 95% 97% or 99% sequence identity with the polypeptide of SEQ ID NO. 1 or 2; Preferably the optional signal peptide has an amino acid sequence with at least 80%, more preferably at least 90%, 95% 97% or 99% sequence identity with the polypeptide of SEQ ID NO 1. Preferably, the signal peptide is present.
a VH domain separated to a VL domain by a linker, said VH and VL contributing to the binding to CD33;
a Hinge derived from Fcgamma ( [symbol] ) RIIIalpha ( [symbol] ) having an amino acid sequence with at least 80%, more preferably at least 90%, 95% 97% or 99% sequence identity with the polypeptide of SEQ ID NO. 3;
a transmembrane domain derived from CD8alpha( [symbol] ) having an amino acid sequence with at least 90%, 91%, 92%, 93%, 94%, 95%, 96%, 97%, 98%, 99% or 100% identity with the polypeptide of SEQ ID NO. 6;
a co-stimulatory signal molecule derived from 4-1BB having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9;

In one embodiment, the present invention provides a CD33 specific chimeric antigen receptor comprising:
a optional signal peptide having an amino acid sequence with at least 80%, more preferably at least 90%, 95% 97% or 99% sequence identity with the polypeptide of SEQ ID NO. 1 or 2; Preferably the optional signal peptide has an amino acid sequence with at least 80%, more preferably at least 90%, 95% 97% or 99% sequence identity with the polypeptide of SEQ ID NO 1. Preferably, the signal peptide is present.
a VH domain separated to a VL domain by a linker, said VH and VL contributing to the binding to CD33;
a Hinge derived from Fcgamma ( [symbol] ) RIIIalpha ( [symbol] ) having an amino acid sequence with at least 80%, more preferably at least 90%, 95% 97% or 99% sequence identity with the polypeptide of SEQ ID NO. 3;
a transmembrane domain (TM) derived from 4-1BB having an amino acid sequence with at least 90%, 91%, 92%, 93%, 94%, 95%, 96%, 97%, 98%, 99% or 100% identity with the polypeptide of SEQ ID NO. 7;
a co-stimulatory signal molecule derived from 4-1BB having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9.

In one embodiment, the present invention provides a CD33 specific chimeric antigen receptor comprising:
a optional signal peptide having an amino acid sequence with at least 80%, more preferably at least 90%, 95% 97% or 99% sequence identity with the polypeptide of SEQ ID NO. 1 or 2; Preferably the optional signal peptide has an amino acid sequence with at least 80%, more preferably at least 90%, 95% 97% or 99% sequence identity with the polypeptide of SEQ ID NO 1. Preferably, the signal peptide is present.
a VH domain separated to a VL domain by a linker, said VH and VL contributing to the binding to CD33;
a Hinge derived from human CD8 alpha chain having an amino acid sequence with at least 80%, more preferably at least 90%, 95% 97% or 99% sequence identity with the polypeptide of SEQ ID NO. 4;
a transmembrane domain derived from CD8alpha( [symbol] ) having an amino acid sequence with at least 90%, 91%, 92%, 93%, 94%, 95%, 96%, 97%, 98%, 99% or 100% identity with the polypeptide of SEQ ID NO. 6;
a co-stimulatory signal molecule derived from 4-1BB having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9;

In one embodiment, the present invention provides a CD33 specific chimeric antigen receptor comprising:
a optional signal peptide having an amino acid sequence with at least 80%, more preferably at least 90%, 95% 97% or 99% sequence identity with the polypeptide of SEQ ID NO. 1 or 2; Preferably the optional signal peptide has an amino acid sequence with at least 80%, more preferably at least 90%, 95% 97% or 99% sequence identity with the polypeptide of SEQ ID NO 1. Preferably, the signal peptide is present.
a VH domain separated to a VL domain by a linker, said VH and VL contributing to the binding to CD33;
a Hinge derived from human CD8 alpha chain having an amino acid sequence with at least 80%, more preferably at least 90%, 95% 97% or 99% sequence identity with the polypeptide of SEQ ID NO. 4;
a transmembrane domain (TM) derived from 4-1BB having an amino acid sequence with at least 90%, 91%, 92%, 93%, 94%, 95%, 96%, 97%, 98%, 99% or 100% identity with the polypeptide of SEQ ID NO. 7;
a co-stimulatory signal molecule derived from 4-1BB having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9.

In one embodiment, the present invention provides a CD33 specific chimeric antigen receptor comprising:
a optional signal peptide having an amino acid sequence with at least 80%, more preferably at least 90%, 95% 97% or 99% sequence identity with the polypeptide of SEQ ID NO. 1 or 2; Preferably the optional signal peptide has an amino acid sequence with at least 80%, more preferably at least 90%, 95% 97% or 99% sequence identity with the polypeptide of SEQ ID NO 1. Preferably, the signal peptide is present.
a VH domain separated to a VL domain by a linker, said VH and VL contributing to the binding to CD33;
a Hinge derived from IgG1 having an amino acid sequence with at least 80%, more preferably at least 90%, 95% 97% or 99% sequence identity with the polypeptide of SEQ ID NO. 5;
a transmembrane domain derived from CD8alpha( [symbol] ) having an amino acid sequence with at least 90%, 91%, 92%, 93%, 94%, 95%, 96%, 97%, 98%, 99% or 100% identity with the polypeptide of SEQ ID NO. 6;
a co-stimulatory signal molecule derived from 4-1BB having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9;

In one embodiment, the present invention provides a CD33 specific chimeric antigen receptor comprising:
a optional signal peptide having an amino acid sequence with at least 80%, more preferably at least 90%, 95% 97% or 99% sequence identity with the polypeptide of SEQ ID NO. 1 or 2; Preferably the optional signal peptide has an amino acid sequence with at least 80%, more preferably at least 90%, 95% 97% or 99% sequence identity with the polypeptide of SEQ ID NO 1. Preferably, the signal peptide is present.
a VH domain separated to a VL domain by a linker, said VH and VL contributing to the binding to CD33;
a Hinge derived from IgG1 having an amino acid sequence with at least 80%, more preferably at least 90%, 95% 97% or 99% sequence identity with the polypeptide of SEQ ID NO. 5;
a transmembrane domain (TM) derived from 4-1BB having an amino acid sequence with at least 90%, 91%, 92%, 93%, 94%, 95%, 96%, 97%, 98%, 99% or 100% identity with the polypeptide of SEQ ID NO. 7;
a co-stimulatory signal molecule derived from 4-1BB having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9.

In one embodiment, the present invention provides at least one of the following CD33 specific CAR having the following sequences - or 80% of the following sequences:

M195-1
SEQ ID NO 48:
MALPVTALLLPLALLLHAARPEVQLQQSGPELVKPGASVKISCKASGYTFTDYNMHWVKQSHGKSLEWIGY
IYPYNGGTGYNQKFKSKATLTVDNSSSTAYMDVRSLTSEDSAVYYCARGRPAMDYWGQGTSVTVSSGG
GGSGGGGSGGGGSDIVLTQSPASLAVSLGQRATISCRASESVDNYGISFMNWFQQKPGQPPKLLIYAA
SNQGSGVPARFSGSGSGTDFSLNIHPMEEDDTAMYFCQQSKEVPWTFGGGTKLEIKGLAVSTISSFFPPGY
QIYIWAPLAGTCGVLLLSLVITLYCKRGRKKLLYIFKQPFMRPVQTTQEEDGCSCRFPEEEEGGCELRVKFSRSADAPA
YQQGQNQLYNELNLGRREEYDVLDKRRGRDPEMGGKPRRKNPQEGLYNELQKDKMAEAYSEIGMKGERRRGKG
HDGLYQGLSTATKDTYDALHMQALPPR
M195-2
SEQ ID NO 49:
MALPVTALLLPLALLLHAARPEVQLQQSGPELVKPGASVKISCKASGYTFTDYNMHWKQSHGKSLEWIGY
IYPYNGGTGYNQKFKSKATLTVDNSSSTAYMDVRSLTSEDSAVYYCARGRPAMDYWGQGTSVTVSSGG
GGSGGGGSGGGGSDIVLTQSPASLAVSLGQRATISCRASESVDNYGISFMNWFQQKPGQPPKLLIYAA
SNQGSGVPARFSGSGSGTDFSLNIHPMEEDDTAMYFCQQSKEVPWTFGGGTKLEIKGLAVSTISSFFPPGY
QIISFFLALTSTALLFLLFFLTLRFSVVKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIGMKGERRRGK
GHDGLYQGLSTATKDTYDALHMQALPPR
M195-3
SEQ ID NO 50:
MALPVTALLLPLALLLHAARPEVQLQQSGPELVKPGASVKISCKASGYTFTDYNMHWVKQSHGKSLEWIGY
IYPYNGGTGYNQKFKSKATLTVDNSSSTAYMDVRSLTSEDSAVYYCARGRPAMDYWGQGTSVTVSSGG
GGSGGGGSGGGGSDIVLTQSPASLAVSLGQRATISCRASESVDNYGISFMNWFQQKPGQPPKLLIYAA
SNQGSGVPARFSGSGSGTDFSLNIHPMEEDDTAMYFCQQSKEVPWTFGGGTKLEIKTTTPAPRPPTPAPTI
ASQPLSLRPEACRPAAGGAVHTRGLDFACDIYIWAPLAGTCGVLLLSLVITLYCKRGRKKLLYIFKQPFMRPVQTTQE
EDGCSCRFPEEEEGGCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
M195-4
SEQ ID NO 51:
MALPVTALLLPLALLLHAARPEVQLQQSGPELVKPGASVKISCKASGYTFTDYNMHWVKQSHGKSLEWIGY
IYPYNGGTGYNQKFKSKATLTVDNSSSTAYMDVRSLTSEDSAVYYCARGRPAMDYWGQGTSVTVSSGG
GGSGGGGSGGGGSDIVLTQSPASLAVSLGQRATISCRASESVDNYGISFMNWFQQKPGQPPKLLIYAA
SNQGSGVPARFSGSGSGTDFSLNIHPMEEDDTAMYFCQQSKEVPWTFGGGTKLEIKTTTPAPRPPTPAPTI
ASQPLSLRPEACRPAAGGAVHTRGLDFACDIISFFLALTSTALLFLLFFLTLRFSVVKRGRKKLLYIFKQPFMRPVQTTQ
EEDGCSCRFPEEEEGGCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
M195-5
SEQ ID NO 52:
MALPVTALLLPLALLLHAARPEVQLQQSGPELVKPGASVKISCKASGYTFTDYNMHWKQSIIGKSLEWIGY
IYPYNGGTGYNQKFKSKATLTVDNSSSTAYMDVRSLTSEDSAVYYCARGRPAMDYWGQGTSVTVSSGG
GGSGGGGSGGGGSDIVLTQSPASLAVSLGQRATISCRASESVDNYGISFMNWFQQKPGQPPKLLIYAA
SNQGSGVPARFSGSGSGTDFSLNIHPMEEDDTAMYFCQQSKEVPWTFGGGTKLEIKEPKSPDKTHTCPPC
PAPPVAGPSVFLFPPKPKDTLMIARTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPGKIYIWAPLAGTCG
VLLLSLVITLYCKRGRKKLLYIFKQPFMRPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEIGMKGERRRGKGHDGLYQGLSTATK
DTYDALHMQALPPR
M195-6
SEQ ID NO 53:
MALPVTALLLPLALLLHAARPEVQLQQSGPELVKPGASVKISCKASGYTFTDYNMHWVKQSHGKSLEWIGY
IYPYNGGTGYNQKFKSKATLTVDNSSSTAYMDVRSLTSEDSAVYYCARGRPAMDYWGQGTSVTVSSGG
GGSGGGGSGGGGSDIVLTQSPASLAVSLGQRATISCRASESVDNYGISFMNWFQQKPGQPPKLLIYAA
SNQGSGVPARFSGSGSGTDFSLNIHPMEEDDTAMYFCQQSKEVPWTFGGGTKLEIKEPKSPDKTHTCPPC
PAPPVAGPSVFLFPPKPKDTLMIARTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPGKIISFFLALTSTALL
FLLFFLTLRFSVVKRGRKKLLYIFKQPFMRPVQTTQEEDGCSCRFIDEEEEGGCELRVKFSRSADAPAYQQGQNQLYN
ELNLGRREEYDVLDKRRGRDPEMGGKPRRKNPQEGLYNELQKDKMAEAYSEIGMKGERRRGKGHDGLYQGLSTA
TKDTYDALHMQALPPR
m2H12-1
SEQ ID NO 54:
MALPVTALLLPLALLLHAARPQVQLQQSGPELVRPGTFVKISCKASGYTFTNYDINWVNQRPGQGLEWIGW
IYPGDGSTKYNEKFKAKATLTADKSSSTAYLQLNNLTSENSAVYFCASGYEDAMDYWGQGTSVTVSSG
GGGSGGGGSGGGGSDIKMTQSPSSMYASLGERVIINCKASQDINSYLSWFQQKPGKSPKTLIYRANRL
VDGVPSRFSGSGSGQDYSLTISSLEYEDMGIYYCLQYDEFPLTFGAGTKLELKRGLAVSTISSFFPPGYQIY
IWAPLAGTCGVLLLSLVITLYCKRGRKKLLYIFKQPFMRPVQTTQEEDGCSCRFPEEEEGGCELRVKFSRSADAPAYQ
QGQNQLYNELNLGRREEYDVLDKRRGRDPEMGGKPRRKNPQEGLYNELQKDKMAEAYSEIGMKGERRRGKGHD
GLYQGLSTATKDTYDALHMQALPPR
m2H12-2
SEQ ID NO 55:
MALPVTALLLPLALLLHAARPQVQLQQSGPELVRPGTFVKISCKASGYTFTNYDINWNQRPGQGLEWIGW
IYPGDGSTKYNEKFKAKATLTADKSSSTAYLQLNNLTSENSAVYFCASGYEDAMDYWGQGTSVTVSSG
GGGSGGGGSGGGGSDIKMTQSPSSMYASLGERVIINCKASQDINSYLSWFQQKPGKSPKTLIYRANRL
VDGVPSRFSGSGSGQDYSLTISSLEYEDMGIYYCLQYDEFPLTFGAGTKLELKRGLAVSTISSFFPPGYQII
SFFLALTSTALLFLLFFLTLRFSVVKRGRKKLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQKDKMAEAYSEIGMKGERRRGKGH
DGLYQGLSTATKDTYDALHMQALPPR
m2H12-3
SEQ ID NO 56:
MALPVTALLLPLALLLHAARPQVQLQQSGPELVRPGTFVKISCKASGYTFTNYDINWVNQRPGQGLEWIGW
IYPGDGSTKYNEKFKAKATLTADKSSSTAYLQLNNLTSENSAVYFCASGYEDAMDYWGQGTSVTVSSG
GGGSGGGGSGGGGSDIKMTQSPSSMYASLGERVIINCKASQDINSYLSWFQQKPGKSPKTLIYRANRL
VDGVPSRFSGSGSGQDYSLTISSLEYEDMGIYYCLQYDEFPLTFGAGTKLELKRTTTPAPRPPTPAPTIAS
QPLSLRPEACRPAAGGAVHTRGLDFACDIYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
m2H12-4
SEQ ID NO 57:
MALPVTALLLPLALLLHAARPQVQLQQSGPELVRPGTFVKISCKASGYTFTNYDINWVNQRPGQGLEWIGW
IYPGDGSTKYNEKFKAKATLTADKSSSTAYLQLNNLTSENSAVYFCASGYEDAMDYWGQGTSVTVSSG
GGGSGGGGSGGGGSDIKMTQSPSSMYASLGERVIINCKASQDINSYLSWFQQKPGKSPKTLIYRANRL
VDGVPSRFSGSGSGQDYSLTISSLEYEDMGIYYCLQYDEFFLTFGAGTKLELKRTTTPAPRPPTPAPTIAS
QPLSLRPEACRPAAGGAVHIRGLDFACDIISFFLALTSTALLFLLFFLTLRFSVVKRGRKKLLYIFKQPFMRPVQTTQEE
DGCSCRFPEEEEGGCELRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
m2H12-5
SEQ ID NO 58:
MALPVTALLLPLALLLHAARPQVQLQQSGPELVRPGTFVKISCKASGYTFTNYDINWVNQRPGQGLEWIGW
IYPGDGSTKYNEKFKAKATLTADKSSSTAYLQLNNLTSENSAVYFCASGYEDAMDYWGQGTSVTVSSG
GGGSGGGGSGGGGSDIKMTQSPSSMYASLGERVIINCKASQDINSYLSWFQQKPGKSPKTLIYRANRL
VDGVPSRFSGSGSGQDYSLTISSLEYEDMGIYYCLQYDEFPLTFGAGTKLELKREPKSPDKTHTCPPCPA
PPVAGPSVFLFPPKPKDTLMIARTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGKIYIWAPLAGTCGVL
LLSLVITLYCKRGRKKLLYIFKQPFMRPVQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGMKGERRRGKGHDGLYQGLSTATKDT
YDALHMQALPPR
m2H12-6
SEQ ID NO 59:
MALPVTALLLPLALLLHAARPQVQLQQSGPELVRPGTFVKISCKASGYTFTNYDINWVNQRPGQGLEWIGW
IYPGDGSTKYNEKFKAKATLTADKSSSTAYLQLNNLTSENSAVYFCASGYEDAMDYWGQGTSVTVSSG
GGGSGGGGSGGGGSDIKMTQSPSSMYASLGERVIINCKASQDINSYLSWFQQKPGKSPKTLIYRANRL
VDGVPSRFSGSGSGQDYSLTISSLEYEDMGIYYCLQYDEFPLTFGAGTKLELKREPKSPDKTHTCPPCPA
PPVAGPSVFLFPPKPKDTLMIARTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGKIISFFLALTSTALLFLL
FFLTLRFSVVKRGRKKLLYIFKQPFMRPVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIGMKGERRRGKGHDGLYQGLSTATKD
TYDALHMQALPPR
DRB2-1
SEQ ID NO 60:
MALPVTALLLPLALLLHAARPEVKLQESGPELVKPGASVKMSCKASGYKFTDYVVHWLKQKPGQGLEWTGY
INPYNDGTKYNEKFKGKATLTSDKSSSTAYMEVSSLTSEDSAVYYCARDYRYEVYGMDYWGQGTSVTV
SSGGGGSGGGGSGGGGSDIVLTQSPTIMSASPGERVTMTCTASSSVNYIHWYQQKSGDSPLRWIFDTS
KVASGVPARFSGSGSGTSYSLTISTMEAEDAATYYCQQWRSYPLTFGDGTRLELKRADAAPTVSGLAVS
TISSFFPPGYQIYIWAPLAGTCGVLLLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR
DRB2-2
SEQ ID NO 61:
MALPVTALLLPLALLLHAARPEVKLQESGPELVKPGASVKMSCKASGYKFTDYVVHWLKQKPGQGLEWIGY
INPYNDGTKYNEKFKGKATLTSDKSSSTAYMEVSSLTSEDSAVYYCARDYRYEVYGMDYWGQGTSVTV
SSGGGGSGGGGSGGGGSDIVLTQSPTIMSASPGERVTMTCTASSSVNYIHWYQQKSGDSPLRWIEDTS
KVASGVPARFSGSGSGTSYSLTISTMEAEDAATYYCQQWRSYPLTFGDGTRLELKRADAAPTVSGLAVS
TISSFFPPGYQIISFFLALTSTALLFLLFFLTLRFSVVKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
DRB2-3
SEQ ID NO 62:
MALPVTALLLPLALLLHAARPEVKLQESGPELVKPGASVKMSCKASGYKFTDYVVHWLKQKPGQGLEWIGY
INPYNDGTKYNEKFKGKATLTSDKSSSTAYMEVSSLTSEDSAVYYCARDYRYEVYGMDYWGQGTSVTV
SSGGGGSGGGGSGGGGSDIVLTQSPTIMSASPGERVTMTCTASSSVNYIHWYQQKSGDSPLRWIEDTS
KVASGVPARFSGSGSGTSYSLTISTMEAEDAATYYCQQWRSYPLTFGDGTRLELKRADAAPTVSTTTPA
PRPPTPAPTIASQPLSLRPEACRPAAGGAVHTRGLDFACDIYIWAPLAGTCGVLLLSLVITLYCKRGRKKLLYIFKQPF
MRPVQTTQEEDGCSCRFPEEEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHMQALPPR
DRB2-4
SEQ ID NO 63:
MALPVTALLLPLALLLHAARPEVKLQESGPELVKPGASVKMSCKASGYKFTDYVVHWLKQKPGQGLEWIGY
INPYNDGTKYNEKFKGKATLTSDKSSSTAYMEVSSLTSEDSAVYYCARDYRYEVYGMDYWGQGTSVTV
SSGGGGSGGGGSGGGGSDIVLTQSPTIMSASPGERVTMTCTASSSVNYIHWYQQKSGDSPLRWIEDTS
KVASGVPARFSGSGSGTSYSLTISTMEAEDAATYYCQQWRSYPLTFGDGTRLELKRADAAPTVSTTTPA
PRPPTPAPTIASQPLSLRPEACRPAAGGAVHTRGLDFACDIISFFLALTSTALLFLLFFLTLRFSVVKRGRKKLLYIFKQPF
MRPVQTTQEEDGCSCRFPEEEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHMQALPPR
DRB2-5
SEQ ID NO 64:
MALPVTALLLPLALLLHAARPEVKIQESGPELVKPGASVKMSCKASGYKETDYVVHWLKQKPGQGLEWIGY
INPYNDGTKYNEKFKGKATLTSDKSSSTAYMEVSSLTSEDSAVYYCARDYRYEVYGMDYWGQGTSVTV
SSGGGGSGGGGSGGGGSDIVLTQSPTIMSASPGERVTMTCTASSSVNYIHWYQQKSGDSPLRWIEDTS
KVASGVPARFSGSGSGTSYSLTISTMEAEDAATYYCQQWRSYPLTFGDGTRLELKRADAAPTVSEPKSP
DKTHTCPPCPAPPVAGPSVFLFPPKPKDTLMIARTPEVICVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNHYTQKSLSLSPGKIYI
WAPLAGTCGVLLLSLVITLYCKRGRKKLLYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKDKMAEAYSEIGMKGERRRGKGHDG
LYQGLSTATKDTYDALHMQALPPR
DRB2-6
SEQ ID NO 65:
MALPVTALLLPLALLLHAARPEVKLQESGPELVKPGASVKMSCKASGYKFTDYVVHWLKQKPGQGLEWIGY
INPYNDGTKYNEKFKGKATLTSDKSSSTAYMEVSSLTSEDSAVYYCARDYRYEVYGMDYWGQGTSVTV
SSGGGGSGGGGSGGGGSDIVLTQSPTIMSASPGERVTMTCTASSSVNYIHWYQQKSGDSPLRWIFDTS
KVASGVPARFSGSGSGTSYSLTISTMEAEDAATYYCQQWRSYPLTFGDGTRLELKRADAAPTVSEPKSP
DKTHTCPPCPAPPVAGPSVFLFPPKPKDTLMIARTPENTICVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNHYTQKSLSLSPGKIISF
FLALTSTALLFLLFFLTLRFSVVKRGRKKLLYIFKQPFMRPVQTTQEEDGCSCRFPEEEEGGCELRVKFSRSADAPAYQ
QGQNQLYNELNLGRREEYDVLDKRRGRDPEMGGKPRRKNPQEGLYNELQKDKMAEAYSEIGMKGERRRGKGHD
GLYQGLSTATKDTYDALHMQALPPR
My9.6-1
SEQ ID NO 66:
MALPVTALLLPLALLLHAARPQVQLQQPGAEVVKPGASVKMSCKASGYTFTSYYIHWIKQTPGQGLEWVGV
IYPGNDDISYNQKFKGKATLTADKSSTTAYMQLSSLTSEDSAVYYCAREVRLRYFDVWGAGTTVTVSS
GGGGSGGGGSGGGGSNIMLTQSPSSLAVSAGEKVTMSCKSSQSVFFSSSQKNYLAWYQQIPGQSPKLL
IYWASTRESGVPDRFTGSGSGTDFTLTISSVQSEDLAIYYCHQYLSSRTFGGGTKLEIKRGLAVSTISSF
FPPGYQIYIWAPLAGTCGVLLLSLVITLYCKRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQEGLYNELQKDKMAEAYSEIGMKGER
RRGKGHDGLYQGLSTATKDTYDALHMQALPPR
My9.6-2
SEQ ID NO 67:
MALPVTALLLPLALLLHAARPQVQLQQPGAEVVKPGASVKMSCKASGYTFTSYYIHWIKQTPGQGLEWVGV
IYPGNDDISYNQKFKGKATLTADKSSTTAYMQLSSLTSEDSAVYYCAREVRLRYFDVWGAGTTVTVSS
GGGGSGGGGSGGGGSNIMLTQSPSSLAVSAGEKVTMSCKSSQSVFFSSSQKNYLAWYQQIPGQSPKLL
IYWASTRESGVPDRFTGSGSGTDFTLTISSVQSEDLAIYYCHQYLSSRTFGGGTKLEIKRGLAVSTISSF
FPPGYQIISFFLALTSTALLFLLFFLTLRFSVVKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
My9.6-3
SEQ ID NO 68:
MALPVTALLLPLALLLHAARPQVQLQQPGAEVVKPGASVKMSCKASGYTFTSYYIHWIKQTPGQGLEWVGV
IYPGNDDISYNQKFKGKATLTADKSSTTAYMQLSSLTSEDSAVYYCAREVRLRYFDVWGAGTTVTVSS
GGGGSGGGGSGGGGSNIMLTQSPSSLAVSAGEKVTMSCKSSQSVFFSSSQKNYLAWYQQIPGQSPKLL
IYWASTRESGVPDRFTGSGSGTDFTLTISSVQSEDLAIYYCHQYLSSRTFGGGTKLEIKRTTTPAPRPPT
PAPTIASQPLSLRPEACRPAAGGAVHTRGLDFACDIYIWAPLAGTCGVLLLSLVITLYCKRGRKKLLYIFKQPFMRPVQ
TTQEEDGCSCRFPEEEEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
My9.6-4
SEQ ID NO 69:
MALPVTALLLPLALLLHAARPQVQLQQPGAEVVKPGASVKMSCKASGYTFTSYYIHWIKQTPGQGLEWVGV
IYPGNDDISYNQKFKGKATLTADKSSTTAYMQLSSLTSEDSAVYYCAREVRLRYFDVWGAGTTVTVSS
GGGGSGGGGSGGGGSNIMLTQSPSSLAVSAGEKVTMSCKSSQSVFFSSSQKNYLAWYQQIPGQSPKLL
IYWASTRESGVPDRFTGSGSGTDFTLTISSVQSEDLAIYYCHQYLSSRTFGGGTKLEIKRTTTPAPRPPT
PAPTIASQPLSLRPEACRPAAGGAVHTRGLDFACDIISFFLALTSTALLFLLFFLTLRFSVVKRGRKKLLYIFKQPFMRPV
QTTQEEDGCSCRFPEEEEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPPR
My9.6-5
SEQ ID NO 70:
MALPVTALLLPLALLLHAARPQVQLQQPGAEVVKPGASVKMSCKASGYTFTSYYIHWIKQTPGQGLEWVGV
IYPGNDDISYNQKFKGKATLTADKSSTTAYMQLSSLTSEDSAVYYCAREVRLRYFDVWGAGTTVTVSS
GGGGSGGGGSGGGGSNIMLTQSPSSLAVSAGEKVTMSCKSSQSVFFSSSQKNYLAWYQQIPGQSPKLL
IYWASTRESGVPDRFTGSGSGTDFTLTISSVQSEDLAIYYCHQYLSSRTFGGGTKLEIKREPKSPDKTH
TCPPCPAPPVAGPSVFLFPPKPKDTLMIARTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGKIYIWAPLA
GTCGVLLLSLVITLYCKRGRKKLLYIFKQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAEAYSEIGMKGERRRGKGHDGLYQGL
STATKDTYDALHMQALPPR
My9.6-6
SEQ ID NO 71:
MALPVTALLLPLALLLHAARPQVQLQQPGAEVVKPGASVKMSCKASGYTFTSYYIHWIKQTPGQGLEWVGV
IYPGNDDISYNQKFKGKATLTADKSSTTAYMQLSSLTSEDSAVYYCAREVRLRYFDVWGAGTTVTVSS
GGGGSGGGGSGGGGSNIMLTQSPSSLAVSAGEKVTMSCKSSQSVFFSSSQKNYLAWYQQIPGQSPKLL
IYWASTRESGVPDRFTGSGSGTDFTLTISSVQSEDLATYYCHQYLSSRTFGGGTKLEIKREPKSPDKTH
TCPPCPAPPVAGPSVFLFPPKPKDTLMIARTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGKIISFFLALT
STALLFLLFFLTLRFSVVKRGRKKLLYIFKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMAEAYSEIGMKGERRRGKGHDGLYQ
GLSTATKDTYDALHMQALPPR.

In one embodiment, the present invention provides primary T cells endowed with at least one of these CD33 specific CAR.

In a preferred embodiment, the present invention provides a CD33 specific chimeric antigen receptor (CAR) with at least 80% of a sequence selected from: SEQ ID No 48 to 71, more preferably at least 80% of SEQ ID NO. 68.
and even more preferably, said CARs preferentially comprise a polypeptide sequence displaying at least 80%, 81%, 82%, 83%, 84%, 85%, 86%, 87%, 88%, 89%, 90%, 91%, 92%, 93%, 94%, 95%, 96%, 97%, 98%, or 99% identity with amino acid sequences consisting of SEQ ID NO:68.

In this embodiment, said CAR preferentially comprises a polypeptide sequence displaying 81% identity with amino acid sequences consisting of SEQ ID NO:68,
said CAR preferentially comprises a polypeptide sequence displaying 82% identity with amino acid sequences consisting of SEQ ID NO:68,
said CAR preferentially comprises a polypeptide sequence displaying 83% identity with amino acid sequences consisting of SEQ ID NO:68,
said CAR preferentially comprises a polypeptide sequence displaying 84% identity with amino acid sequences consisting of SEQ ID NO:68,
said CAR preferentially comprises a polypeptide sequence displaying 85% identity with amino acid sequences consisting of SEQ ID NO:68,
said CAR preferentially comprises a polypeptide sequence displaying 86% identity with amino acid sequences consisting of SEQ ID NO:68,
said CAR preferentially comprises a polypeptide sequence displaying 87% identity with amino acid sequences consisting of SEQ ID NO:68,
said CARs preferentially comprises a polypeptide sequence displaying 88% identity with amino acid sequences consisting of SEQ ID NO:68,
said CAR preferentially comprises a polypeptide sequence displaying 89% identity with amino acid sequences consisting of SEQ ID NO:68,
said CAR preferentially comprises a polypeptide sequence displaying 90% identity with amino acid sequences consisting of SEQ ID NO:68,
said CAR preferentially comprises a polypeptide sequence displaying 91% identity with amino acid sequences consisting of SEQ ID NO:68,
said CAR preferentially comprises a polypeptide sequence displaying 92% identity with amino acid sequences consisting of SEQ ID NO:68,
said CAR preferentially comprises a polypeptide sequence displaying 93% identity with amino acid sequences consisting of SEQ ID NO:68,
said CAR preferentially comprises a polypeptide sequence displaying 94% identity with amino acid sequences consisting of SEQ ID NO:68,
said CAR preferentially comprises a polypeptide sequence displaying 95% identity with amino acid sequences consisting of SEQ ID NO:68,
said CAR preferentially comprises a polypeptide sequence displaying 96% identity with amino acid sequences consisting of SEQ ID NO:68,
said CAR preferentially comprises a polypeptide sequence displaying 97% identity with amino acid sequences consisting of SEQ ID NO:68
said CAR preferentially comprises a polypeptide sequence displaying 98% identity with amino acid sequences consisting of SEQ ID NO:68,
said CAR preferentially comprises a polypeptide sequence displaying 99% identity with amino acid sequences consisting of SEQ ID NO:68,

In one embodiment, said CAR is having 100% identity with amino acid sequences consisting of SEQ ID NO:68.

The present invention encompasses a CAR having a percentage of identity as described herein (from 80% to 99%) with any one of the sequences from SEQ ID 48 to SEQ ID 71.

The present invention provides a CD33 specific CAR comprising
(a) an extra cellular ligand binding-domain comprising VH and VL from a monoclonal anti-CD33 antibody, optionally humanized
(b) a CD80 hinge,
(c) a CD8α transmembrane domain and
(d) a cytoplasmic domain including a CD3 zeta signaling domain and a co-stimulatory domain from 4-1BB.

The present invention provides a CD33 specific CAR comprising
(a) an extra cellular ligand binding-domain comprising VH and VL from a monoclonal anti-CD33 antibody, optionally humanized
(b) a FcγRIIIα hinge,
(c) a CD8α transmembrane domain and
(d) a cytoplasmic domain including a CD3 zeta signaling domain and a co-stimulatory domain from 4-1BB

### Polynucleotides, Vectors:

The present invention also relates to polynucleotides, vectors encoding any one of the above described CARs according to the invention.

The present invention also relates to a polynucleotide encoding an anti-CD33 CAR according to the invention, to a vector, preferably to a lentiviral vector, comprising at least one of said polynucleotide encoding an anti-CD33 CAR according to the invention.

The polynucleotide may consist in an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell).

In a particular embodiment, the different nucleic acid sequences can be included in one polynucleotide or vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptide into the secretory pathway of a host cell, a secretory signal sequence (also known as a leader sequence, prepro sequence or pre sequence) is provided in polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). In a preferred embodiment the signal peptide comprises the amino acid sequence SEQ ID NO: 1 and 2.

Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

Methods of Engineering Immune Cells Endowed with CARs:

The present invention encompasses the method of preparing immune cells for immunotherapy comprising introducing ex-vivo into said immune cells the polynucleotides or vectors encoding one of the CD33 CAR as previously described.

In a preferred embodiment, said polynucleotides are included in lentiviral vectors in view of being stably expressed in the immune cells.

According to further embodiments, said method further comprises the step of genetically modifying said cell to make them more suitable for allogeneic transplantation.

According to a first aspect, the immune cell can be made less allogeneic, for instance, by inactivating at least one gene expressing one or more component of T-cell receptor (TCR) as described in WO 2013/176915, which can be combined with the inactivation of a gene encoding or regulating HLA or β2m protein expression. Accordingly, the risk of graft versus host syndrome and graft rejection is significantly reduced.

According to another aspect, at least one gene allowing the expression of one or more component of CD33 is inactivated in the CD33 specific CAR immune cells of the invention. Inhibiting CD33 surface expression in cells expressing an anti-CD33 CAR is part of a method for preparing the anti-CD33 CAR expressing immune cells of the invention.

A general method is described in WO 2013/176915. Inactivation of CD33 gene can be combined with the activation/or inactivation of a gene encoding or regulating CD33 expression such as sialic acid binding Ig-like lectins (Cao H, Crocker P R. Evolution of CD33-related siglecs: regulating host immune functions and escaping pathogen exploitation? Immunology. 2011 January; 132(1):18-26) so that cell surface expression of CD33 the anti-CD33 CAR expressing immune cells is inhibited and consequently do not alter the survival or inhibit the activity the anti-CD33 CAR expressing neighboring cells.

According to another aspect, the immune cells can be further genetically engineered to improve their resistance to immunosuppressive drugs or chemotherapy treatments, which are used as standard care for treating CD33 positive malignant cells. For instance, CD52 and glucocorticoid receptors (GR), which are drug targets of Campath (alemtuzumab) and glucocorticoids treatments, can be inactivated to make the cells resistant to these treatments and give them a competitive advantage over patient's own T-cells not endowed with specific CD33 CARs. Expression of CD3 gene can also be suppressed or reduced to confer resistance to Teplizumab, which is another immune suppressive drug. Expression of HPRT can also be suppressed or reduced according to the invention to confer resistance to 6-thioguanine, a cytostatic agent commonly used in chemotherapy especially for the treatment of acute lymphoblasic leukemia. Expression of the “GLI1” gene may be reduced.

According to further aspect of the invention, the immune cells can be further manipulated to make them more active or limit exhaustion, by inactivating genes encoding proteins that act as “immune checkpoints” that act as regulators of T-cells activation, such as PDCD1 or CTLA-4. Examples of genes, which expression could be reduced or suppressed are indicated in Table 9.

TABLE 9
List of genes encoding immune checkpoint proteins.

| | | Genes that can be inactivated |
|---|---|---|
| Pathway | | In the pathway |
| Co-inhibitory | CTLA4 | CTLA4, PPP2CA, PPP2CB, PTPN6, |
| receptors | (CD152) | PTPN22 |
| | PDCD1 (PD-1, | PDCD1 |
| | CD279) | |
| | CD223 (lag3) | LAG3 |
| | HAVCR2 | HAVCR2 |
| | (tim3) | |
| | BTLA(cd272) | BTLA |
| | CD160(by55) | CD160 |
| | IgSF family | TIGIT |
| | | CD96 |
| | | CRTAM |
| | LAIR1(cd305) | LAIR1 |
| | SIGLECs | SIGLEC7 |
| | | SIGLEC3 |
| | | SIGLEC9 |
| | CD244(2b4) | CD244 |
| Death receptors | TRAIL | TNFRSF10B, TNFRSF10A, CASP8, |
| | | CASP10, CASP3, CASP6, CASP7 |
| | FAS | FADD, FAS |
| Cytokine | TGF-beta | TGFBRII, TGFBRI, SMAD2, SMAD3, |
| signalling | signaling | SMAD4, SMAD10, SKI, SKIL, TGIF1 |
| | IL10 signalling | IL10RA, IL10RB, HMOX2 |
| | IL6 signalling | IL6R, IL6ST |
| Prevention | | CSK, PAG1 |
| of TCR | | |
| signalling | | SIT1 |
| Induced Treg | induced Treg | FOXP3 |
| Transcription | transcription | PRDM1 (=blimp1, heterozygotes mice |
| factors | factors | control chronic viral infection better |
| controlling | controlling | than wt or conditional KO) |
| exhaustion | exhaustion | |
| | | BATF |
| Hypoxia | iNOS induced | GUCY1A2, GUCY1A3, GUCY1B2, |
| mediated | guanylated | GUCY1B3 |
| tolerance | cyclase | |

In a preferred embodiment said method of further engineering the immune cells involves introducing into said T cells polynucleotides, in particular mRNAs, encoding specific rare-cutting endonuclease to selectively inactivate the genes, as those mentioned above, by DNA cleavage. In a more preferred embodiment said rare-cutting endonucleases are TALE-nucleases or Cas9 endonuclease. TAL-nucleases have so far proven higher specificity and cleavage efficiency over the other types of rare-cutting endonucleases, making them the endonucleases of choice for producing of the engineered immune cells on a large scale with a constant turn-over.

### Delivery Methods

The different methods described above involve introducing CAR into a cell. As non-limiting example, said CAR can be introduced as transgenes encoded by one plasmid vector. Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

### Engineered Immune Cells

The present invention also relates to isolated cells or cell lines susceptible to be obtained by said method to engineer cells. In particular said isolated cell comprises at least one CAR as described above. In another embodiment, said isolated cell comprises a population of CARs each one comprising different extracellular ligand binding domains. In particular, said isolated cell comprises exogenous polynucleotide sequence encoding CAR. Genetically modified immune cells of the present invention are activated and proliferate independently of antigen binding mechanisms.

In the scope of the present invention is also encompassed an isolated immune cell, preferably a T-cell obtained according to any one of the methods previously described. Said immune cell refers to a cell of hematopoietic origin functionally involved in the initiation and/or execution of innate and/or adaptative immune response. Said immune cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+T-lymphocytes and CD8+T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T-cell according to the method previously described. Modified cells resistant to an immunosuppressive treatment and susceptible to be obtained by the previous method are encompassed in the scope of the present invention.

As a preferred embodiment, the present invention provides T-cells or a population of T-cells endowed with a CD33 CAR as described above, that do not express functional TCR and that a reactive towards CD33 positive cells, for their allogeneic transplantation into patients.

As a more preferred embodiment, the present invention provides the engineered immune cells according to the invention comprising T-cells or a population of T-cells endowed with a CD33 CAR as described above, that do neither express a functional TCR nor CD33, and are reactive towards CD33 positive cells, for their allogeneic transplantation into patients.

As an even more preferred embodiment, the present invention provides the engineered immune cells according to the invention comprising T-cells or a population of T-cells endowed with a CD33 CAR, said CD33 CAR comprising a polypeptide structure selected from V1, V3 and V5, said polypeptide structure comprising an extra cellular ligand binding-domain comprising VH and VL from a monoclonal anti-CD33 antibody, a hinge selected from a FcRIIIα hinge, CD8α hinge, and IgG1 hinge, a CD8α transmembrane domain and a cytoplasmic domain including a CD3 zeta signaling domain and a co-stimulatory domain from 4-1BB.

In one embodiment, the engineered immune cells according to the invention comprise a specific CD33 CAR comprising a monoclonal anti-CD33 antibody is selected from M195, m2 h12, DRB2, and My9.6, or from M195, m2 h12, and My9.6, and optionally humanized.

In a more preferred embodiment, said CD33 CAR comprises a polypeptide selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO:70, or comprise a polypeptide selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, SEQ ID NO:70.

In an even more preferred embodiment, T-cells endowed with a CD33 CAR, do not express functional TCR and CD33, and said CD33 CAR comprises a polypeptide of SEQ ID NO: 68, and optionally humanized.

In one even more preferred embodiment, T-cells or a population of T-cells endowed with a CD33 CAR, in particular anti-CD33 CAR expressing T cells that do neither express functional TCR nor CD33, comprise a polypeptide sequence having at least 80%, 81%, 82%, 83%, 84%, 85%, 86%, 87%, 88%, 89%, 90%, 91%, 92%, 93%, 94%, 95%, 96%, 97%, 98% or 99% identity with any one of an amino acid sequence selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, SEQ ID NO:70, or having at least 80%, 81%, 82%, 83%, 84%, 85%, 86%, 87%, 88%, 89%, 90%, 91%, 92%, 93%, 94%, 95%, 96%, 97%, 98% or 99% identity with any one of an amino acid sequence selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, SEQ ID NO:70 or having at least 80%, 81%, 82%, 83%, 84%, 85%, 86%, 87%, 88%, 89%, 90%, 91%, 92%, 93%, 94%, 95%, 96%, 97%, 98% or 99% identity with SEQ ID NO: 68.

In this even more preferred embodiment, T-cells endowed with a CD33 CAR, do not express functional TCR and CD33 and comprise a polypeptide sequence having at least 80%, 81%, 82%, 83%, 84%, 85%, 86%, 87%, 88%, 89%, 90%, 91%, 92%, 93%, 94%, 95%, 96%, 97%, 98% or 99% identity with an amino acid sequence selected from the group consisting of SEQ ID NO: 50, SEQ ID NO: 52, SEQ ID NO: 56, SEQ ID NO: 58, SEQ ID NO: 68, SEQ ID NO:70, or a polypeptide sequence having at least 80%, 81%, 82%, 83%, 84%, 85%, 86%, 87%, 88%, 89%, 90%, 91%, 92%, 93%, 94%, 95%, 96%, 97%, 98% or 99% identity with an amino acid sequence selected from the group consisting of SEQ ID NO: 50, SEQ ID NO: 56, SEQ ID NO: 68 or a polypeptide sequence having at least 80%, 81%, 82%, 83%, 84%, 85%, 86%, 87%, 88%, 89%, 90%, 91%, 92%, 93%, 94%, 95%, 96%, 97%, 98% or 99% identity with SEQ ID NO: 68.

Preferably, said anti-CD33 CAR expressing T cell is a TCRalpha KO and CD33 KO T cell and is resistant to at least one drug used for the treatment of AML.

An anti-CD33 CAR expressing T cell according to the invention represents isolated immune T cells endowed with anti-CD33 CAR, preferably TCRalpha KO and/or CD33 KO immune T cells endowed with anti-CD33 CAR cells and more preferably TCRalpha KO and/or CD33 KO immune T cells endowed with anti-CD33 CAR cells that are resistant to at least one drug used for the treatment of AML. Preferably, an anti-CD33 CAR expressing T cell expressed in T cells according to the invention comprises an anti-CD33 CAR having one of the sequences selected from SEQ ID NO 48 to SEQ ID NO 71, more preferably an anti-CD33 CAR having at least 80% identity with the SEQ ID NO 48 to SEQ ID NO 71 and even more preferably at least 80% identity with SEQ ID NO 68. An Engineered immune cell (or anti-CD33 CAR-expressing T cells) according to the invention means any one of the engineered immune cell according to the invention, described above.

### Activation and Expansion of T Cells

Whether prior to or after genetic modification of the T cells, even if the genetically modified immune cells of the present invention are activated and proliferate independently of antigen binding mechanisms, the immune cells, particularly T-cells of the present invention can be further activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

Generally, the T cells of the invention are expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

### Therapeutic Applications

In another embodiment, isolated cell obtained by the different methods or cell line derived from said isolated cell as previously described can be used as a medicament. In another embodiment, said medicament can be used for treating cancer, particularly for the treatment of B-cell lymphomas and leukemia in a patient in need thereof. In another embodiment, said isolated cell according to the invention or cell line derived from said isolated cell can be used in the manufacture of a medicament for treatment of a cancer in a patient in need thereof.

In another aspect, the present invention relies on methods for treating patients in need thereof, said method comprising at least one of the following steps:
(a) providing an immune-cell obtainable by any one of the methods previously described;
(b) Administrating said transformed immune cells to said patient,

On one embodiment, said T cells of the invention can undergo robust in vivo T cell expansion and can persist for an extended amount of time.

In a preferred aspect, the present invention relies on methods for treating patients in need thereof, said method comprising at least one of the following steps:
(a) Providing an immune-cell obtainable by any one of the methods of the invention to prepare an anti-CD33 expressing CAR immune cell, (or any of the engineered immune cell of the invention)
(b) Administrating said anti-CD33 expressing CAR immune cells to said patient; optionally, said CD33 CAR T cell of the invention can undergo robust in vivo T cell expansion and can persist for an extended amount of time, in particular can bind to CD33 for an extended amount of time.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

In one embodiment, an anti-CD33 CAR expressing T cell according to the invention is provided for its use as a medicament.

In a particular embodiment, said anti-CD33 CAR expressing T cell according to the invention provided for its use as a medicament, comprises a polypeptide selected from SEQ ID NO 19, SEQ ID NO 20, SEQ ID NO 21, SEQ ID NO 22, SEQ ID NO 27, SEQ ID NO 28, SEQ ID NO 29, SEQ ID NO 30, SEQ ID NO 35, SEQ ID NO 36, SEQ ID NO 37, SEQ ID NO 38 and more preferably a polypeptide having at least 80% identity with a polypeptide selected from a polypeptide of SEQ ID NO 19, SEQ ID NO 20, SEQ ID NO 21, SEQ ID NO 22, SEQ ID NO 27, SEQ ID NO 28, SEQ ID NO 29, SEQ ID NO 30, SEQ ID NO 35, SEQ ID NO 36, SEQ ID NO 37, SEQ ID NO 38,

Even more preferably, said anti-CD33 CAR expressing T cell according to the invention provided for its use as a medicament, comprises a polypeptide selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO:70, or comprise a polypeptide selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, SEQ ID NO:70; any of these CAR may be humanized.

In one embodiment, an anti-CD33 CAR expressing T cell according to the invention provided for its use as a medicament, comprises a polypeptide of SEQ ID NO: 68 or a polypeptide having at least 80% identity with a polypeptide of SEQ ID NO: 68, optionally humanized.

In other words, the invention is related to an anti-CD33 CAR expressing T cell according to the invention comprising a polypeptide having at least 80% identity with any one of the polypeptide selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68 and SEQ ID NO:70, optionally humanized for its use as a medicament.

Engineered immune cells that can be used as a medicament or with the disclosed methods are described in the previous sections. Preferably Engineered immune cells may comprise primary T cells, wherein a CD33 expression in altered, a TCR expression is altered, optionally resistant to at least one drug used to treat an hematological cancer.

In general, said medicament can be used for treating a CD33-expressing cell-mediated pathological condition or a condition characterized by the direct or indirect activity of a CD33-expressing cell, ie a condition linked to the detrimental activity of CD33-expressing cells.

Said medicament can be used to treat patients diagnosed wherein a pre-malignant or malignant cancer condition characterized by CD33-expressing T cells, especially by an overabundance of CD33-expressing T cells. Such conditions are found in hematologic cancers, such as leukemia or malignant lymphoproliferative disorders such as B-cell lymphoproliferative disorders.

Another example of CD33-expressing cell-mediated pathological condition or a condition characterized by the direct or indirect activity of a CD33-expressing cell that can be treated with an anti-CD33 CAR expressing T cell according to the invention is Alzheimer disease.

A Lymphoproliferative disorder can be lymphoma, in particular multiple myeloma, non-Hodgkin's lymphoma, Burkitt's lymphoma, and follicular lymphoma (small cell and large cell).

Any one of the CD33-mediating or CD33-involving disease in particular malignant lymphoproliferative disorder or leukemia may be treated and the health condition of the patient suffering said pathological condition may be improved, with the anti-CD33 CAR-expressing T cells of the present invention.

In a preferred embodiment, the cancer that may be treated using the anti-CD33 CAR-expressing T cells of the present invention is leukemia, a disease associated to leukemia or a complication thereof, in particular AML, an AML subtype, AML-related complication, and AML-related conditions.

A leukemia that can also be prevented or treated using the anti-CD33 CAR-expressing T cells of the present invention can be acute myelogenous leukemia (AML), chronic myelogenous leukemia, melodysplastic syndrome, acute lymphoid leukemia, acute lymphoblastic leukemia, chronic lymphoid leukemia, and myelodysplastic syndrome.

AML or AML subtypes that may be treated using the anti-CD33 CAR-expressing T cells of the present invention may be in particular, acute myeloblastic leukemia, minimally differentiated acute myeloblastic leukemia, acute myeloblastic leukemia without maturation, acute myeloblastic leukemia with granulocytic maturation, promyelocytic or acute promyelocytic leukemia (APL), acute myelomonocytic leukemia, myelomonocytic together with bone marrow eosinophilia, acute monoblastic leukemia (M5a) or acute monocytic leukemia (M5b), acute erythroid leukemia, including erythroleukemia (M6a) and very rare pure erythroid leukemia (M6b), acute megakaryoblastic leukemia (M7), acute basophilic leukemia, acute panmyelosis with myelofibrosis, whether involving CD33-positive cells.

Subtypes of AML that may be treated using the anti-CD33 CAR-expressing T cells of the present invention also include, AML with t (8;21) (q22;q22), (AML1/ETO), AML with inv (16) (p13;q22) or t (16;16) (p13;q22), (CBFβ/MYH11), AML with t (15;17) (q22;q12), (PML/RARα) and variants, AML with t(9;11)(p22;q23), (MLLT3/MLL), AML with t(6;9)(p23;q34) (DEK/NUP214), AML with inv(3)(q21q26) or t(3;3)(q21;q26), (RPN1/EVI1), AML with t(1;22)(p13;q13) (RBM15/MKL1) (megakaryocytic), AML with myelodysplasia-related changes including AML arising from prior MDS or MDS/MPN, AML with an MDS-related cytogenetic abnormality, and AML with multilineage dysplasia, alkylating agent/radiation related AML, AML, minimally differentiated (also known as AML-M0), AML without maturation (also known as AML-M1), AML with maturation (also known as AML-M2), Acute myelomonocytic leukemia (also known as AML-M4), Acute monoblastic/monocytic leukemia (also known as AML-M5), Acute erythroid leukemia (also known as AML-M6) and pure erythroid leukemia.

Accordingly, AML classified as AML with specific genetic abnormalities are conditions that may be treated using the anti-CD33 CAR-expressing T cells of the present invention. Classification is based on the ability of karyotype to predict response to induction therapy, relapse risk, survival.

Accordingly, AML that may be treated using the anti-CD33 CAR-expressing T cells of the present invention may be AML with a translocation between chromosomes 8 and 21, AML with a translocation or inversion in chromosome 16, AML with a translocation between chromosomes 9 and 11, APL (M3) with a translocation between chromosomes 15 and 17, AML with a translocation between chromosomes 6 and 9, AML with a translocation or inversion in chromosome 3, AML (megakaryoblastic) with a translocation between chromosomes 1 and 22.

The present invention is particularly useful for the treatment of AML associated with these particular cytogenetic markers.

The present invention also provides an anti-CD33 CAR expressing T cell for the treatment of patients with specific cytogenetic subsets of AML, such as patients with t(15;17)(q22;q21) identified using all-trans retinoic acid (ATRA) and for the treatment of patients with t(8;21)(q22;q22) or inv(16)(p13q22)/t(16;16)(p13;q22) identified using repetitive doses of high-dose cytarabine.

Preferably, the present invention provides an anti-CD33 CAR expressing T cell for the treatment of patients with aberrations, such as −5/del(5q), −7, abnormalities of 3q, or a complex karyotype, who have been shown to have inferior complete remission rates and survival.

### Group of Patients

In a preferred embodiment, the invention provides a medication for leukemia in particular for AML in patients over 60 years, in patients of less than 20 years, in particular in children.

In a more preferred embodiment, the present invention provides a pediatric treatment, in particular a pediatric treatment against AML, or AML-related diseases or complications.

In still another preferred embodiment, the present invention is used as a treatment in AML patients with low, poor or unfavorable status that is to say with a predicted survival of less than 5 years survival rate. In this group, patients suffering AML with the following cytogenetic characteristics: −5; 5q; −7; 7q−;11q23; non t(9;11); inv(3); t(3;3); t(6;9); t(9;22) is associated with poor-risk status (Byrd J. C. et al., Dec. 15, 2002; Blood: 100 (13) and is especially contemplated to be treated according to the present invention or with an object of the present invention.

In one embodiment, the anti-CD33 CAR expressing T cell of the present invention may be used as a treatment in case of AML relapse, or in case of refractory or resistant AML. Preferably, T cells comprising at least one humanized anti-CD33 CAR of the invention comprising or consisting of SEQ ID NO. 1 and of a polypeptide selected from a polypeptide having at least 80% to 100% identity with SEQ ID NO 19, SEQ ID NO 20, SEQ ID NO 21, SEQ ID NO 22, SEQ ID NO 27, SEQ ID NO 28, SEQ ID NO 29, SEQ ID NO 30, SEQ ID NO 35, SEQ ID NO 36, SEQ ID NO 37, SEQ ID NO 38, or a combination thereof, are used in patients with AML relapse, or with refractory or resistant AML, more preferably, in combination with at least one other anti-cancer drug.

In another preferred embodiment, said least one anti-CD33 CAR T cell of the invention, is used for preventing cancer cells development occurring in particular after anti-cancer treatment, during bone marrow depletion or before bone marrow transplantation, after bone marrow destruction.

### AML Complications

In one particular embodiment the invention provides a medicament that improves the health condition of a patient, in particular a patient undergoing a complication related to AML. More preferably, said engineered anti-CD33 CAR expressing T cell of the invention is expressing at least one anti-CD33 CAR comprising a SEQ ID NO 1 and a polypeptide having at least 80% identity with SEQ ID NO 19, SEQ ID NO 20, SEQ ID NO 21, SEQ ID NO 22, SEQ ID NO 27, SEQ ID NO 28, SEQ ID NO 29, SEQ ID NO 30, SEQ ID NO 35, SEQ ID NO 36, SEQ ID NO 37, SEQ ID NO 38, or a combination thereof, and is used as a medicament for the treatment of a complication related to AML.

In one particular embodiment the invention provides a medicament that improves the health condition of a patient suffering from AML, in particular a patient undergoing a complication related to AML, said medicament comprising an anti-CD33 CAR expressing T cell of the invention comprising a polypeptide having at least 80% identity with a polypeptide selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, and SEQ ID NO:70.

A complication or disease related to AML may include a preceding myelodysplasia phase, secondary leukemia, in particular secondary AML, high white blood cell count, and absence of Auer rods. Among others, leukostasis and involvement of the central nervous system (CNS), Hyperleukocytosis, residual disease, are also considered as a complication or disease related to AML.

### AML Associated Diseases

In one embodiment, the present invention also provides an anti-CD33 CAR expressing T cell for the treatment of a pathological condition related to AML. Preferably, the present invention provides a cell expressing at least one anti-CD33 CAR comprising a polypeptide having at least 80% identity with a polypeptide selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, and SEQ ID NO:70. for the treatment of a pathological condition related to AML.

The present invention provides a medicament for AML related myeloid neoplasms, for acute myeloid leukemia and myelodysplastic syndrome, treatment of relapsed or refractory acute myeloid leukemia, a treatment of relapsed or refractory acute promyelocytic leukemia in adults, a treatment for acute promyeloid leukaemia, a treatment of acute myeloid leukemia in adults over 60 years.

According to another aspect, the present invention provides a composition for the treatment of AML associated diseases, in particular hematologic malignancy related to AML.

Hematologic malignancy related to AML conditions include myelodysplasia syndromes (MDS, formerly known as “preleukemia”) which are a diverse collection of hematological conditions united by ineffective production (or dysplasia) of myeloid blood cells and risk of transformation to AML.

In another embodiment, the invention provides a medicament according to the invention that improves the health state of a patient suffering multiple myeloma.

Other pathological conditions or genetic syndromes associated with the risk of AML can be improved with the adequate use of the present invention, said genetic syndromes include Down syndrome, trisomy, Fanconi anemia, Bloom syndrome, Ataxia-telangiectasia, Diamond-Blackfan anemia, Schwachman-Diamond syndrome, Li-Fraumeni syndrome, Neurofibromatosis type 1, Severe congenital neutropenia (also called Kostmann syndrome).

In one embodiment the present invention provides an anti-CD33 CAR expressing T cell for the treatment of Alzheimer's disease. The present invention provides cells that can be used with the disclosed methods are described in the previous section, preferably cells are anti-CD33 CAR expressing T cells and more preferably TCR and CD33 KO anti-CD33 CAR expressing T cells. Said treatment can be used to treat patients diagnosed wherein a pre-malignant or malignant cancer condition characterized by CD33-expressing cells, especially by an overabundance of CD33-expressing cells. Such conditions are found in hematologic cancers, such as leukemia or malignant lymphoproliferative disorders.

Leukemia can be acute myelogenous leukemia, chronic myelogenous leukemia, melodysplastic syndrome, acute lymphoid leukemia, chronic lymphoid leukemia, and myelodysplastic syndrome.

Lymphoproliferative disorder can be lymphoma, in particular multiple myeloma, non-Hodgkin's lymphoma, Burkitt's lymphoma, and follicular lymphoma (small cell and large cell).

Cancers that may be treated may comprise nonsolid tumors (such as hematological tumors, including but not limited to pre-B ALL (pediatric indication), adult ALL, mantle cell lymphoma, diffuse large B-cell lymphoma and the like. Types of cancers to be treated with the CARs of the invention include, but are not limited leukemia or lymphoid malignancies. Adult tumors/cancers and pediatric tumors/cancers are also included.

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

### Compositions

The present invention provides a composition comprising an anti-CD33 expressing T cells according to the invention and a pharmaceutically acceptable vehicle.

In one embodiment said composition is provided for use as a medicament.

In another embodiment said composition is provided for use as a medicament for the treatment of conditions characterized by CD33-expressing cells, in particular by an overabundance of CD33-expressing cells. Such conditions are found in hematologic cancers, such as leukemia or malignant lymphoproliferative disorders such as B-cell lymphoproliferative disorders.

According to one aspect, the present invention provides a composition comprising an anti-CD33 expressing T cells according to the invention and a pharmaceutically acceptable vehicle for the treatment of CD33+ cell-mediated diseases. These CD33+ cell mediated diseases include a pre-malignant or malignant cancer condition, inflammation, autoimmune diseases, Alzheimer disease.

In one aspect, a CD33-expressing hematologic cancer cell that can be treated using a composition according to the invention may be a CD33-expressing hematologic cancer stem cell including but not limited to CD33-expressing cancer cell in leukemia (such as acute myelogenous leukemia (AML), chronic myelogenous leukemia, acute lymphoid leukemia, chronic lymphoid leukemia and myelodysplasia syndrome) and malignant lymphoproliferative conditions, including lymphoma (such as multiple myeloma, non-Hodgkin's lymphoma, Burkitt's lymphoma, and small cell- and large cell-follicular lymphoma), or a complication thereof.

A composition comprising an anti-CD33 expressing T cells according to the invention and a pharmaceutically acceptable vehicle is provided for use in a method of reducing the amount, inhibiting the proliferation and/or activity of CD33-expressing hematologic cancer cells in a patient. An exemplary method includes contacting a population of cells comprising a CD33-expressing cell with a CD 33 CART cell of the invention that binds to the CD33-expressing cell.

In a more specific aspect, the present invention provides a composition comprising an anti-CD33 expressing T cells according to the invention and a pharmaceutically acceptable vehicle for its use, in particular in a method for inhibiting the proliferation or reducing the population of cancer cells expressing CD33 in a patient, the methods comprising contacting the CD33-expressing cancer cell population with a CD 33 CART cell of the invention that binds to the CD33-expressing cell, binding of a CD 33 CART cell of the invention to the CD33-expressing cancer cell resulting in the destruction of the CD33-expressing cancer cells.

In certain aspects, the composition comprising a CD 33 CART cell of the invention reduces the quantity, number, amount or percentage of cells and/or cancer cells by at least 25%, at least 30%, at least 40%, at least 50%, at least 65%, at least 75%, at least 85%, at least 95%, or at least 99% (to undetectable level) in a subject with or animal model for myeloid leukemia or another cancer associated with CD33-expressing cells, relative to a negative control.

The present invention also provides a method for preventing, treating and/or managing a disorder or condition associated with CD33-expressing cells (e.g., associated with a hematologic cancer, AML), the methods comprising administering to a subject in need a composition comprising a CD 33 CART cell of the invention that binds to the CD33-expressing cell, in particular a composition comprising an anti-CD33 expressing T cells according to the invention and a pharmaceutically acceptable vehicle. In one aspect, the subject is a human. Non-limiting examples of disorders associated with CD33-expressing cells include inflammatory disorders (such as allergies, Inflammatory bowel diseases BD, Alzheimer disease) and cancers (such as hematological cancers, in particular AML or AML complications).

The present invention also provides a composition for its use or a method for treating a disease comprising the T cell expressing an anti-CD33 CAR of the invention and a pharmaceutically acceptable vehicle, preferably, said anti-CD33 CAR comprising a SEQ ID NO. 1 and a polypeptide having at least 80% to 100% identity with a polypeptide selected from SEQ ID NO 19, SEQ ID NO 20, SEQ ID NO 21, SEQ ID NO 22, SEQ ID NO 27, SEQ ID NO 28, SEQ ID NO 29, SEQ ID NO 30, SEQ ID NO 35, SEQ ID NO 36, SEQ ID NO 37, SEQ ID NO 38, and a combination thereof, and a pharmaceutically acceptable carrier or vehicle. In one aspect, the disease is a diseases as described herein, in particular a hematologic cancer, more particularly a stem cell cancer including but is not limited to leukemia (such as acute myelogenous leukemia (AML), chronic myelogenous leukemia, acute lymphoid leukemia, chronic lymphoid leukemia and myelodysplasia syndrome) and malignant lymphoproliferative conditions, including lymphoma (such as multiple myeloma, non-Hodgkin's lymphoma, Burkitt's lymphoma, and small cell- and large cell-follicular lymphoma), or a complication thereof.

The present invention also provides a composition comprising an anti-CD33 expressing T cells according to the invention and a pharmaceutically acceptable vehicle for its use in a method for inhibiting the proliferation or reducing a CD33-expressing cell population or activity in a patient. An exemplary method includes contacting a population of cells comprising a CD33-expressing cell with a CD 33 CART cell of the invention that binds to the CD33-expressing cell, preferably said anti-CD33 CAR comprises a SEQ ID NO. 1 and a polypeptide having at least 80% to 100% identity with a polypeptide selected from SEQ ID NO 19, SEQ ID NO 20, SEQ ID NO 21, SEQ ID NO 22, SEQ ID NO 27, SEQ ID NO 28, SEQ ID NO 29, SEQ ID NO 30, SEQ ID NO 35, SEQ ID NO 36, SEQ ID NO 37, SEQ ID NO 38, and a combination thereof.

The present invention also provides a composition comprising an anti-CD33 expressing T cells according to the invention and a pharmaceutically acceptable vehicle for its use in a method for preventing, treating and/or managing a disorder or condition associated with CD33-expressing cells (e.g., associated with a hematologic cancer), the methods comprising administering to a subject in need a composition of the invention, wherein said anti-CD33 CAR comprises a SEQ ID NO. 1 and a polypeptide having at least 80% to 100% identity with a polypeptide selected from SEQ ID NO 19, SEQ ID NO 20, SEQ ID NO 21, SEQ ID NO 22, SEQ ID NO 27, SEQ ID NO 28, SEQ ID NO 29, SEQ ID NO 30, SEQ ID NO 35, SEQ ID NO 36, SEQ ID NO 37, SEQ ID NO 38, and a combination thereof, that binds to the CD33-expressing cell. In one aspect, the subject is a human. Non-limiting examples of disorders associated with CD33-expressing cells include autoimmune disorders (such as lupus), inflammatory disorders (such as allergies, IBD, and asthma) and cancers (such as hematological cancers, in particular AML or AML complications).

The present invention also provides a composition for its use comprising the T cell expressing an anti-CD33 CAR of the invention and a pharmaceutically acceptable vehicle, or a method for treating a disease comprising it use, preferably, said anti-CD33 CAR comprising a polypeptide having at least 80% to 100% identity with a polypeptide selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, and SEQ ID NO:70. In one aspect, the disease is a diseases as described herein, in particular a hematologic cancer, more particularly a stem cell cancer including but is not limited to leukemia (such as acute myelogenous leukemia (AML), chronic myelogenous leukemia, acute lymphoid leukemia, chronic lymphoid leukemia and myelodysplasia syndrome) and malignant lymphoproliferative conditions, including lymphoma (such as multiple myeloma, non-Hodgkin's lymphoma, Burkitt's lymphoma, and small cell- and large cell-follicular lymphoma), or a complication thereof.

The present invention also provides a composition comprising an anti-CD33 expressing T cells according to the invention and a pharmaceutically acceptable vehicle for its use in a method for inhibiting the proliferation or reducing a CD33-expressing cell population or activity in a patient. An exemplary method includes contacting a population of cells comprising a CD33-expressing cancer cell with a CD 33 CAR T cell of the invention that binds to the CD33-expressing cell, preferably said anti-CD33 CAR comprises a polypeptide having at least 80% identity with a sequence selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, and SEQ ID NO:70.

The present invention also provides a composition comprising an anti-CD33 expressing T cells according to the invention and a pharmaceutically acceptable vehicle for its use in a method for preventing, treating and/or managing a disorder or condition associated with CD33-expressing cells (e.g., associated with a hematologic cancer), the methods comprising administering to a subject in need a composition of the invention, wherein said anti-CD33 CAR comprises a polypeptide having at least 80% to 100% identity with a polypeptide selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, and SEQ ID NO:70.

In one aspect, the subject is a human. Non-limiting examples of disorders associated with CD33-expressing cells include autoimmune disorders (such as lupus), inflammatory disorders (such as allergies, IBD, and asthma) and cancers (such as hematological cancers, in particular AML or AML complications).

The present invention also provides a composition comprising an anti-CD33 expressing T cells according to the invention and a pharmaceutically acceptable vehicle for its use in a method for preventing, treating and/or managing a disease associated with CD33-expressing cells, the method comprising administering to a subject in need a composition of the invention that binds to the CD33-expressing cell. In this embodiment, said anti-CD33 CAR comprises a polypeptide having at least 80% to 100% identity with a polypeptide selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, and SEQ ID NO:70. In one aspect, the subject is a human. Non-limiting examples of diseases associated with CD33-expressing cells include Acute Myeloid Leukemia (AML), myelodysplasia, B-cell Acute Lymphoid Leukemia, T-cell Acute Lymphoid Leukemia, hairy cell leukemia, blastic plasmacytoid dendritic cell neoplasm, chronic myeloid leukemia, Hodgkin lymphoma.

The present invention provides a composition comprising an anti-CD33 expressing T cells according to the invention and a pharmaceutically acceptable vehicle for its use in a method for treating or preventing relapse of cancer associated with CD33-expressing cells, the method comprising administering to a subject in need thereof a composition according to the invention, wherein said anti-CD33 CAR comprises a polypeptide having at least 80% to 100% identity with a polypeptide selected from SEQ ID NO: 48, SEQ ID NO: 50, SEQ ID NO: 52, SEQ ID NO: 54, SEQ ID NO: 56, SEQ ID NO: 58, SEQ ID NO: 66, SEQ ID NO: 68, and SEQ ID NO:70 that binds to the CD 33− expressing cell. In another aspect, the methods comprise administering to the subject in need thereof an effective amount of composition according to the invention in combination with an effective amount of another therapy.

In one aspect, the invention provides compositions and methods for treating subjects that have undergone treatment for a disease or disorder associated with elevated expression levels of CD 19, and exhibits a disease or disorder associated with elevated levels of CD33.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

### Other Definitions

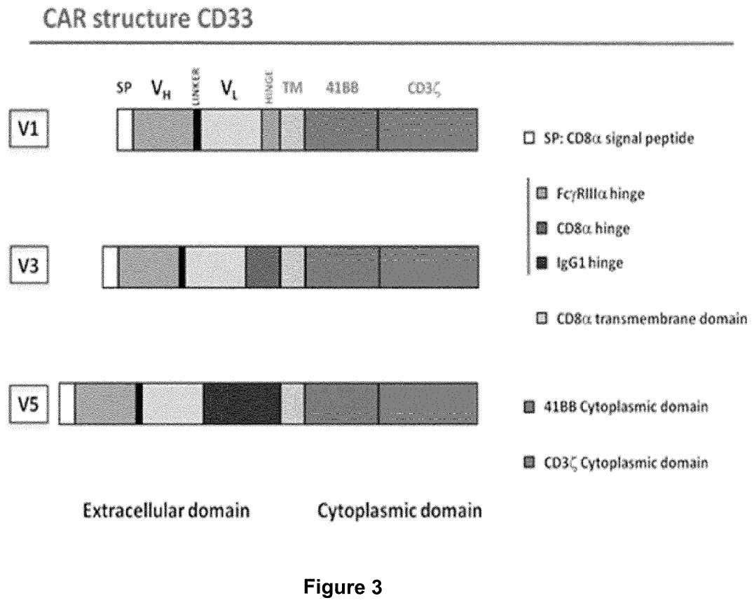

Unless otherwise specified, “a,” “an,” “the,” and “at least one” are used interchangeably and mean one or more than one.—Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
By chimeric antigen receptor (CAR) is intended molecules that combine a binding domain against a component present on the target cell, for example an antibody-based specificity for a desired antigen (e.g., tumor antigen) with a T cell receptor-activating intracellular domain to generate a chimeric protein that exhibits a specific anti-target cellular immune activity. Generally, CAR consists of an extracellular single chain antibody (scFvFc) fused to the intracellular signaling domain of the T cell antigen receptor complex zeta chain (scFvFc:ζ) and have the ability, when expressed in T cells, to redirect antigen recognition based on the monoclonal antibody's specificity. One example of CAR used in the present invention is a CAR directing against CD33 antigen and can comprise as non-limiting example the amino acid sequences: SEQ ID NO: 19 to 42 and preferably the amino acid sequences from SEQ ID NO 48 to 71.
By V1 structure is intended molecules that combine
   a CD8alpha signal peptide,
   a VH domain separated to a VL domain by a linker, said VH and VL contributing to the binding to CD33,
   a Hinge from Fcgamma (γ) RIIIalpha (α)
   a transmembrane domain derived from CD8alpha(α)
   a cytoplasmic domain derived from 41BB and CD3 zeta (ζ)
By V2 structure is intended molecules with a V1 structure and wherein the transmembrane domain derived from 41BB
By V3 structure is intended molecules that combine
   a CD8alpha signal peptide,
   a VH domain separated to a VL domain by a linker, said VH and VL contributing to the binding to CD33,
   a Hinge from CD8alpha (α)
   a transmembrane domain derived from CD8alpha(α)
   a cytoplasmic domain derived from 41BB and CD3 zeta (ζ)
By V4 structure is intended molecules with a V3 structure and wherein the transmembrane domain derived from 41BB.
By V5 structure is intended molecules that combine
   a CD8alpha signal peptide,
   a VH domain separated to a VL domain by a linker, said VH and VL contributing to the binding to CD33,
   a Hinge from IgG1 (α)
   a transmembrane domain derived from CD8alpha(α)
   a cytoplasmic domain derived from 41BB and CD3 zeta (ζ).
By V6 structure is intended molecules with a V5 structure and wherein the transmembrane domain derived from 41BB.
   The CAR structures of the invention are illustrated in FIG. 2, preferably in FIG. 3.

The term “chemotherapy” refers to any therapy using a chemical, in particular those used against cancer. —The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 12 base pairs (bp) in length, more preferably of 14-55 bp. Rare-cutting endonucleases significantly increase HR by inducing DNA double-strand breaks (DSBs) at a defined locus (Perrin, Buckle et al. 1993; Rouet, Smih et al. 1994; Choulika, Perrin et al. 1995; Pingoud and Silva 2007). Rare-cutting endonucleases can for example be a homing endonuclease (Paques and Duchateau 2007), a chimeric Zinc-Finger nuclease (ZFN) resulting from the fusion of engineered zinc-finger domains with the catalytic domain of a restriction enzyme such as FokI (Porteus and Carroll 2005), a Cas9 endonuclease from CRISPR system (Gasiunas, Barrangou et al. 2012; Jinek, Chylinski et al. 2012; Cong, Ran et al. 2013; Mali, Yang et al. 2013) or a chemical endonuclease (Eisenschmidt, Lanio et al. 2005; Arimondo, Thomas et al. 2006). In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer 2005). Such chemical endonucleases are comprised in the term “endonuclease” according to the present invention.
By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALE-nuclease have been already described and used to stimulate gene targeting and gene modifications (Boch, Scholze et al. 2009; Moscou and Bogdanove 2009; Christian, Cermak et al. 2010; Li, Huang et al. 2011). Engineered TAL-nucleases are commercially available under the trade name TALEN™ (Cellectis, 8 rue de la Croix Jarry, 75013 Paris, France).

The rare-cutting endonuclease according to the present invention can also be a Cas9 endonuclease. Recently, a new genome engineering tool has been developed based on the RNA-guided Cas9 nuclease (Gasiunas, Barrangou et al. 2012; Jinek, Chylinski et al. 2012; Cong, Ran et al. 2013; Mali, Yang et al. 2013) from the type II prokaryotic CRISPR (Clustered Regularly Interspaced Short palindromic Repeats) adaptive immune system (see for review (Sorek, Lawrence et al. 2013)). The CRISPR Associated (Cas) system was first discovered in bacteria and functions as a defense against foreign DNA, either viral or plasmid. CRISPR-mediated genome engineering first proceeds by the selection of target sequence often flanked by a short sequence motif, referred as the proto-spacer adjacent motif (PAM). Following target sequence selection, a specific crRNA, complementary to this target sequence is engineered. Trans-activating crRNA (tracrRNA) required in the CRISPR type II systems paired to the crRNA and bound to the provided Cas9 protein. Cas9 acts as a molecular anchor facilitating the base pairing of tracRNA with cRNA (Deltcheva, Chylinski et al. 2011). In this ternary complex, the dual tracrRNA:crRNA structure acts as guide RNA that directs the endonuclease Cas9 to the cognate target sequence. Target recognition by the Cas9-tracrRNA:crRNA complex is initiated by scanning the target sequence for homology between the target sequence and the crRNA. In addition to the target sequence-crRNA complementarity, DNA targeting requires the presence of a short motif adjacent to the protospacer (protospacer adjacent motif—PAM). Following pairing between the dual-RNA and the target sequence, Cas9 subsequently introduces a blunt double strand break 3 bases upstream of the PAM motif (Garneau, Dupuis et al. 2010).

Rare-cutting endonuclease can be a homing endonuclease, also known under the name of meganuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Preferred homing endonuclease according to the present invention can be an I-CreI variant.
By “delivery vector” or “delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adenoassociated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as nonlimiting example, that are able to integrate the genome of a target cell. At the opposite by “non-integrative lentiviral vectors (or NILV)” is meant efficient gene delivery vectors that do not integrate the genome of a target cell through the action of the virus integrase.
Delivery vectors and vectors can be associated or combined with any cellular permeabilization techniques such as sonoporation or electroporation or derivatives of these techniques.
By cell or cells is intended any eukaryotic living cells, primary cells and cell lines derived from these organisms for in vitro cultures.
By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines.

As non-limiting examples cell lines can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

All these cell lines can be modified by the method of the present invention to provide cell line models to produce, express, quantify, detect, study a gene or a protein of interest; these models can also be used to screen biologically active molecules of interest in research and production and various fields such as chemical, biofuels, therapeutics and agronomy as non-limiting examples.
by “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “variant(s)”, it is intended a repeat variant, a variant, a DNA binding variant, a TALE-nuclease variant, a polypeptide variant obtained by mutation or replacement of at least one residue in the amino acid sequence of the parent molecule.
by “functional variant” is intended a catalytically active mutant of a protein or a protein domain; such mutant may have the same activity compared to its parent protein or protein domain or additional properties, or higher or lower activity.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.
“signal-transducing domain” or “co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory Ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a Tcell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor.

A “co-stimulatory signal” as used herein refers to a signal, which in combination with primary signal, such as TCR/CD3 ligation, leads to T cell proliferation and/or upregulation or downregulation of key molecules.

The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. Thus examples of cell surface markers that may act as ligands include those associated with viral, bacterial and parasitic infections, autoimmune disease and cancer cells.

The term “subject” or “patient” as used herein includes all members of the animal kingdom including non-human primates and humans.

The terms “drug used to treat a cancer, in particular AML” refers to medicament used for the treatment of cancer, in particular AML and are described for example in document PCT/EP2015/055848.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

### General Methods:

### Inactivation of Specific Gene(s) in Primary T Cells

Inactivation of specific gene(s) in primary T cells may be performed before or after CAR introduction into T cells.

At least one gene, one gene or two genes may be inactivated in one step or in successive steps. In a preferred embodiment two genes may be inactivated at once, preferably TCRalpha gene and CD33 gene.

In general, heterodimeric nuclease, in particular TALE-Nuclease targeting two long sequences (called half targets) separated by a spacer within a target gene is designed and produced.

Each TALE-nuclease construct may be cloned in an appropriate mammalian expression vector. mRNA encoding TALE-nuclease cleaving a targeted genomic sequence may be synthesized from plasmid carrying the coding sequence downstream a promoter.

Cells are purified T cells preactivated with anti-CD3/CD28 coated beads. Cells are transfected with each of the 2 mRNAs encoding both half TALE-nucleases, in particular both half TALE-nucleases and spacer.

Cells may be reactivated with soluble anti-CD28 to measure cell proliferation and the activation marker CD25 detected to assess the activation state of the cells.

### Chimeric Antigen Receptors

### Nucleic Acids—Vectors

An acid nucleic (mRNA or lentiviral vector) encoding an anti-CD33 CAR of the invention is constructed according to the architecture designed in FIG. 2 or FIG. 3.

Anti-CD33 CAR lentiviral vectors may be prepared for example as previously described in WO2013176915, WO2013176916, or in WO2014130635, and incorporated herein by reference. Lentiviral vectors are produced by Vectalys SA (Toulouse, France).

CAR mRNAs may be produced using T7 mRNA polymerase and transfections done using Cytopulse technology.

### Screening and Selection of Anti-CD33 CAR

### Primary T-Cell Cultures

T cells are purified from Buffy coat samples provided by EFS (Etablissement Français du Sang, Paris, France) using Ficoll gradient density medium. The PBMC layer is recovered and T cells purified using a commercially available T-cell enrichment kit. Purified T cells are activated in X-Vivo™-15 medium (Lonza) using Human IL-2 and Dynabeads Human T activator CD3/CD28.

CAR mRNA Transfection

Transfections of CAR mRNAs encoding the different CAR constructs are performed at Day 4 or Day 11 after T-cell purification and activation.

T-cell transduction with recombinant lentiviral vectors allowing the expression of CAR Transduction of T-cells with recombinant lentiviral vectors are carried out three days after T-cell purification/activation. Lentiviral vectors are produced by Vectalys SA (Toulouse, France), by transfecting genomic and helper plasmids in HEK-293 cells. Transductions may be carried out at various multiplicity of infection (MOI), in particular at a MOI of 5. CAR detection at the surface of T-cells is performed using a recombinant protein consisting on the extracellular domain of the human CD33 protein fused together with a murine IgG1 Fc fragment (produced by LakePharma).

Binding of this protein to the CAR molecule is detected with a PE-conjugated secondary antibody (Jackson Immunoresearch) targeting the mouse Fc portion of the protein, and

### Degranulation Assay (CD107a Mobilization)

T-cells are incubated together with an equal amount of cells expressing various levels of the CD33 protein. Co-cultures are maintained for at least 6 hours. CD107a staining is performed during cell stimulation, by the addition of a fluorescent anti-CD107a antibody at the beginning of the co-culture. After the 6 h incubation period, cells are stained with a fixable viability dye and fluorochrome-conjugated anti-CD8 and analyzed by flow cytometry. The degranulation activity is determined as the % of CD8+/CD107a+ cells, and by determining the mean fluorescence intensity signal (MFI) for CD107a staining among CD8+ cells.

Degranulation assays are carried out 24 h after mRNA transfection.

### IFN Gamma Release Assay

24 h after mRNA transfection, anti-CD33 CAR expressing T-cells are incubated together with cell lines expressing various levels of the CD33 protein for 24 hours at 37° C. The supernatants are recovered and IFN gamma detection in the cell culture supernatants is done by ELISA assay.

### Cytotoxicity Assay

CD33 CAR expressing T-cells are incubated together with target cells (expressing various levels of CD33) or (CD33neg) cells in the same well. Target CD33+ and control CD33-target cells are labelled with fluorescent intracellular dyes (eg. CFSE or Cell Trace Violet), before co-culture with for 4 hours at 37° C. After this incubation period, cells are labelled with a fixable viability dye and analyzed by flow cytometry. Viability of each cellular population (target cells or CD33neg control cells) is determined and the % of specific cell lysis is calculated. Cytotoxicity assays are carried out 48 h after mRNA transfection.

### Anti-Tumor Mouse Model

Immuno deficient NOG mice are intravenously (iv) injected with CD33 expressing −Luciferase cells. Optionally, mice receive an anti-cancer treatment before injection with anti-CD33 CAR+ T-cells. Mice are then iv injected (eg either 2 or 7 days after injection of the tumor cell line) with different doses of anti-CD33 CAR+ T-cells to be tested, or with T-cells that were not transduced with the CAR lentiviral vector. Bioluminescent signals are determined at the day of T-cell injection (DO), at D7, 14, 21, 28 and 40 after T-cell injection in order to follow tumoral progression in the different animals.

Having generally described this invention, a further understanding can be obtained by reference to certain specific examples, which are provided herein for purposes of illustration only, and are not intended to be limiting unless otherwise specified.

### EXAMPLES

### Construction of CD33 CAR Using Various Anti-CD33 Antibody Fragments

Different architectures of CAR were designed (FIG. 3) namely V1 to V6. As a first step, 4 different scFv's from M195, DRB2, m2H12 and My9.6 antibody were constructed to generate Chimeric Antigen Receptors (CARs) (SEQ ID NO 48 to SEQ ID NO 71) in T cells.

### Example 1: Proliferation of TCRalpha Inactivated Cells Expressing a CD33-CAR

### Primary T-Cell Cultures

T cells were purified from various buffy coat samples provided by EFS (Etablissement Français du Sang, Paris, France) using Ficoll gradient density medium. The PBMC layer was recovered and T cells were purified using a commercially available T-cell enrichment kit (Stem Cell Technologies). Purified T cells were activated in X-Vivo™-15 medium (Lonza) supplemented with 20 ng/mL Human IL-2, 5% Human Serum, and Dynabeads Human T activator CD3/CD28 at a bead:cell ratio 1:1 (Life Technologies). After activation cells were grown and maintained in X-Vivo™-15 medium (Lonza) supplemented with 20 ng/mL Human IL-2 and 5% Human Serum.

CAR mRNA Transfection

Transfections of each CD33 CAR generated using various anti-CD33 antibody fragments were done at Day 4 or Day 11 after T-cell purification and activation. 5 million cells were transfected with 15 μg of mRNA encoding the different CAR constructs. CAR mRNAs were produced using the mMESSAGE mMACHINE T7 Kit (Life Technologies) and purified using RNeasy Mini Spin Columns (Qiagen). Transfections were done using Cytopulse technology, by applying two 0.1 mS pulses at 3000V/cm followed by four 0.2 mS pulses at 325V/cm in 0.4 cm gap cuvettes in a final volume of 200 μl of “Cytoporation buffer T” (BTX Harvard Apparatus). Cells were immediately diluted in X-Vivo™-15 media (Lonza) and incubated at 37° C. with 5% CO₂. IL-2 was added 2 h after electroporation at 20 ng/mL.

### Primary T Cells Transduction

### T-Cell Transduction

Transduction of T-cells with recombinant lentiviral vectors expression the antiCD33 CAR were carried out three days after T-cell purification/activation. Lentiviral vectors produced by Vectalys SA (Toulouse, France) were used. Anti-CD33 CAR detection at the surface of T-cells is performed using a recombinant protein consisting on the extracellular domain of the human CD33 protein fused together with a murine IgG1 Fc fragment (produced by LakePharma). Binding of this protein to the anti-CD33 CAR molecule is detected with a PE-conjugated secondary antibody (Jackson Immunoresearch) targeting the mouse Fc portion of the protein, and analyzed by flow cytometry.

Heterodimeric TALE-nuclease targeting two 17-bp long sequences (called half targets) separated by an 15-bp spacer within T-cell receptor alpha constant chain region (TRAC) gene were designed and produced. Each half target is recognized by repeats of the half TALE-nucleases listed in Table 10.

TABLE 10
TAL-nucleases targeting TCRalpha gene

| Target | Target sequence | Repeat sequence | Half TALE-nuclease |
|---|---|---|---|
| TRAC_T01 | TTGTCCCACAGATATCC | Repeat TRAC_T01-L | TRAC _T01-L TALEN |
| | Agaaccctgaccctg | (SEQ ID NO: 44) | (SEQ ID NO: 46) |
| | CCGTGTACCAGCTGAGA | Repeat TRAC_T01-R | TRAC_ T01-R TALEN |
| | (SEQ ID NO: 43) | (SEQ ID NO: 45) | (SEQ ID NO: 47) |

Each TALE-nuclease construct was subcloned using restriction enzyme digestion in a mammalian expression vector under the control of the T7 promoter. mRNA encoding TALE-nuclease cleaving TRAC genomic sequence were synthesized from plasmid carrying the coding sequence downstream from the T7 promoter.

Purified T cells preactivated during 72 hours with anti-CD3/CD28 coated beads were transfected with each of the 2 mRNAs encoding both half TRAC_T01 TALE-nucleases. 48 hours post-transfection, different groups of T cells from the same donor were respectively transduced with a lentiviral vector encoding one of the CD33 CAR previously described (SEQ ID NO: 19 to 42). 2 days post-transduction, CD3_{NEG} cells were purified using anti-CD3 magnetic beads and 5 days post-transduction cells were reactivated with soluble anti-CD28 (5 μg/ml).

Cell proliferation was followed for up to 30 days after reactivation by counting cell 2 times per week. Increased proliferation in TCR alpha inactivated cells expressing the CD33 CARs, especially when reactivated with anti-CD28, was observed compared to non-transduced cells.

To investigate whether the human T cells expressing the CD33CAR display activated state, the expression of the activation marker CD25 are analyzed by FACS 7 days post transduction. The purified cells transduced with the lentiviral vector encoding CD33 CAR assayed for CD25 expression at their surface in order to assess their activation in comparison with the non-transduced cells. Increased CD25 expression is expected both in CD28 reactivation or no reactivation conditions.

The present invention provided a CD33 specific CAR T cell wherein the level of TCR at the cell surface was below detected.

### Example 2

### Inactivation of the CD33 Gene

To inactivate the CD33 gene, heterodimeric TALE-nuclease targeting two sequences (called Sequence bound by TALEN Left and Sequence bound by TALEN Right, see table 11) separated by a 10 or 15-bp spacer within CD33 gene were designed and produced as described below. Each half target is recognized by repeats of the half TALE-nucleases listed in Table 11. The constructs were then introduced into T cells together with and at the same time that those designed to inactivate the TCR alpha gene.

TABLE 11
TAL-nucleases targeting TCRalpha gene

| Target | Target sequences |
|---|---|
| TALEN1_CD33_Exon3 | TGCATCCCCTCTTTCTCCTCACTAGACTTGACCCACAGGCCCAA (SEQ ID NO: 72) |
| TALEN2_CD33_Exon3 | TTCTCCTCACTAGACTTGACCCACAGGCCCAAAATCCTCATCCCTGGCA (SEQ ID |
| | NO: 73) |
| TALEN1_CD33_Exon4 | TCCTCTCCTAGATGTTCCACAGAACCCAACAACTGGTATCTTTCCAGGA (SEQ ID |
| | NO: 74) |
| TALEN2_CD33_Exon4 | TCCTAGATGTTCCACAGAACCCAACAACTGGTATCTTTCCAGGA (SEQ ID NO: 75) |

| Sequence bound by TALEN Left | Spacer | Sequence bound by TALEN Right |
|---|---|---|
| TGCATCCCCTCTTTCTC (SEQ ID | CTCACTAGAC (SEQ ID | TTGACCCACAGGCCCAA (SEQ ID |
| NO: 76) | NO: 80) | NO: 84) |
| TTCTCCTCACTAGACTT (SEQ ID | GACCCACAGGCCCAA (SEQ | AATCCTCATCCCTGGCA (SEQ ID |
| NO: 77) | ID NO: 81) | NO: 85) |
| TCCTCTCCTAGATGTTC (SEQ ID | CACAGAACCCAACAA (SEQ | CTGGTATCTTTCCAGGA (SEQ ID |
| NO: 78) | ID NO: 82) | NO: 86) |
| TCCTAGATGTTCCACAG (SEQ ID | AACCCAACAA (SEQ ID | CTGGTATCTTTCCAGGA (SEQ ID |
| NO: 79) | NO: 83) | NO: 87) |

In the resulting cells, the extracellular domain of CD33 is truncated. Double staining of the resulting cells and analysis by flow cytometry indicated a drop in cell surface expression of CD33 and of TCR as compared to control (mock-transfected) cells.

Alternatively, purified TCR KO T cells preactivated during 72 hours with anti-CD3/CD28 coated beads may be used for CD33 inactivation.

To investigate the activated state of the human TCR KO and CD33 KO T cells expressing the CD33 CAR, the expression of the activation marker CD25 are analyzed by FACS 7 days post transduction.

Anti-CD33 CARs T cells and anti-CD33 CARs T cells wherein TCR alpha gene and/or CD33 gene is inactivated and wherein CAR is corresponding to architectures V1, V3 and V5, can be produced.

Cells are designated as “T cells” or “CD33 CAR”, for convenience.

### Example 3

### Degranulation Activity of CD33 CAR Generated Using Various Anti-CD33 Antibody Fragments

The activity of the constructs V1, V3 and V5 of M195, m2H12 and My9.6 as described above was first determined upon transient expression in human primary T-cells.

### Degranulation Assay (CD107a Mobilization)

T-cells were then incubated in 96-well plates (40,000 cells/well), together with an equal amount of cells expressing various levels of the CD33 protein. Co-cultures were maintained in a final volume of 100 μl of X-Vivo™-15 medium (Lonza) for 6 hours at 37° C. with 5% CO₂. CD107a staining was done during cell stimulation, by the addition of a fluorescent anti-CD107a antibody at the beginning of the co-culture, together with 1 μg/ml of anti-CD49d, 1 μg/ml of anti-CD28, and 1× Monensin solution. After the 6 h incubation period, cells were stained with a fixable viability dye and fluorochrome-conjugated anti-CD8 and analyzed by flow cytometry. The degranulation activity was determined as the % of CD8+/CD107a+ cells, and by determining the mean fluorescence intensity signal (MFI) for CD107a staining among CD8+ cells. Degranulation assays were carried out 24 h after mRNA transfection.

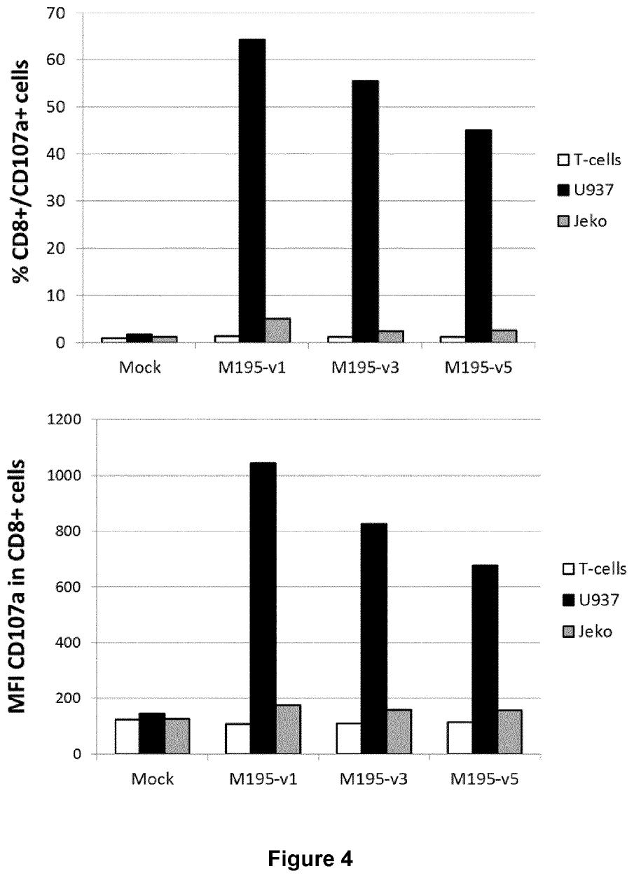

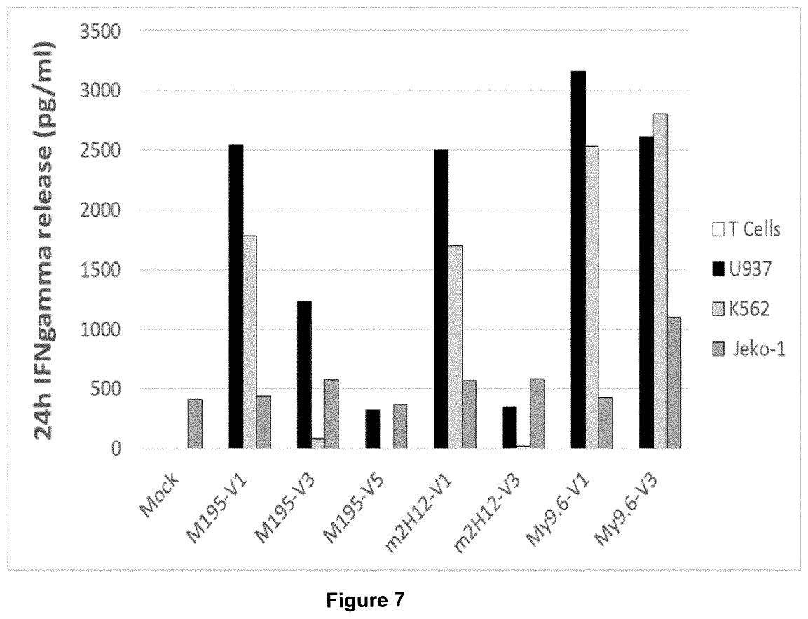

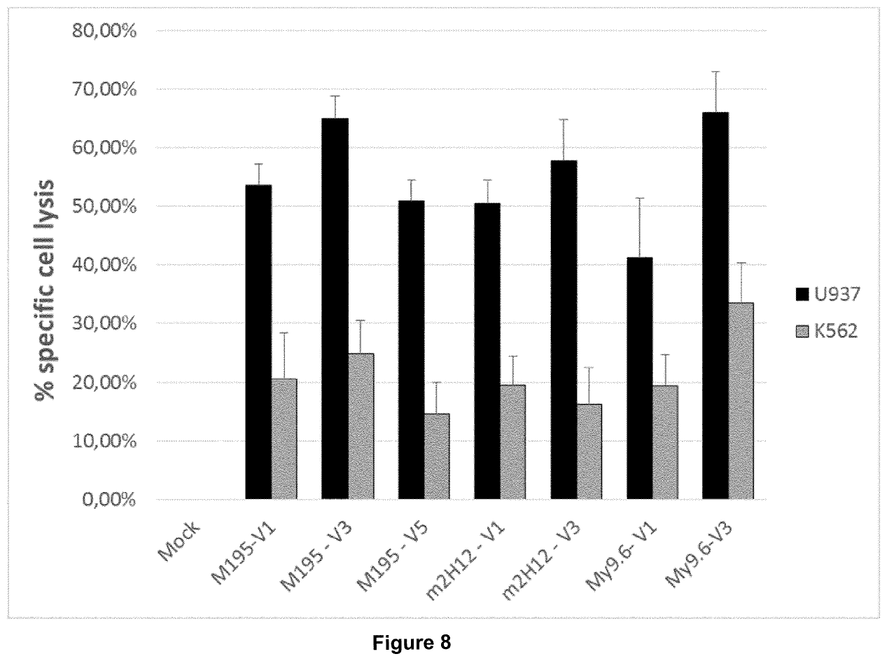

Among the CAR molecules generated as illustrated in FIG. 3, 9 were tested for their degranulation activity (FIG. 4 to FIG. 6) and 7 of them were selected for further activity tests: interferon gamma release and cell lysis (FIG. 7 and FIG. 8).

Degranulation activity of each of the CAR comprising a M195, m2H12 or My9.6 scFv within one of the three architectures (v1: FcgRIII-hinge/CD8-transmembrane, v3: CD8-hinge/CD8-transmembrane, or v5: IgG1-hinge/CD8-transmembrane), was measured when CAR+ T-cells were co-cultured for 6 hours with CD33 expressing cells (U937), or with cells expressing undetectable level CD33 (Jeko or Jeko-1). The results are illustrated in FIG. 4 to FIG. 6.

FIG. 4 shows degranulation activity (% CD8/CD107a+ cells and MFI CD107a in CD8+ cells) of the M195 scFv with the three architectures (v1: FcgRIII-hinge/CD8-transmembrane, v3: CD8-hinge/CD8-transmembrane, v5: IgG1-hinge/CD8-transmembrane), when CAR+ T-cells were co-cultured for 6 hours with CD33 expressing cells (U937), or with cells that do not express CD33 (Jeko). The data is presented as a percentage (%) of degranulation and mean fluorescence activity (MFI).

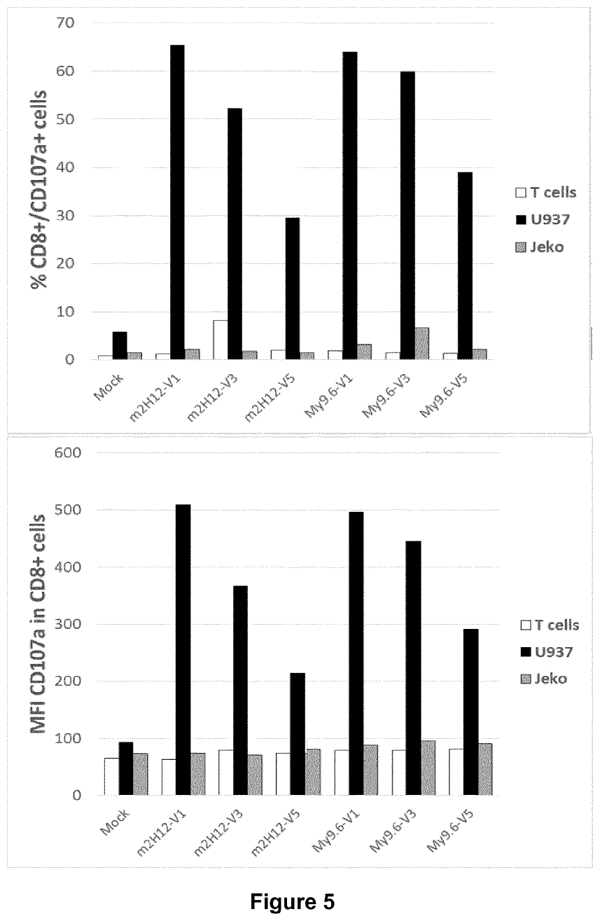

FIG. 5 shows degranulation activity (% CD8/CD107a+ cells and MFI CD107a in CD8+ cells) of the m2H12 and My9.6 scFvs with the three architectures (v1: FcgRIII-hinge/CD8-transmembrane, v3: CD8-hinge/CD8-transmembrane, v5: IgG1-hinge/CD8-transmembrane), when CAR+ T-cells were co-cultured for 6 hours with CD33 expressing cells (U937), or with cells that do not express CD33 (Jeko). The data is presented as a percentage (%) of degranulation and mean fluorescence activity (MFI).

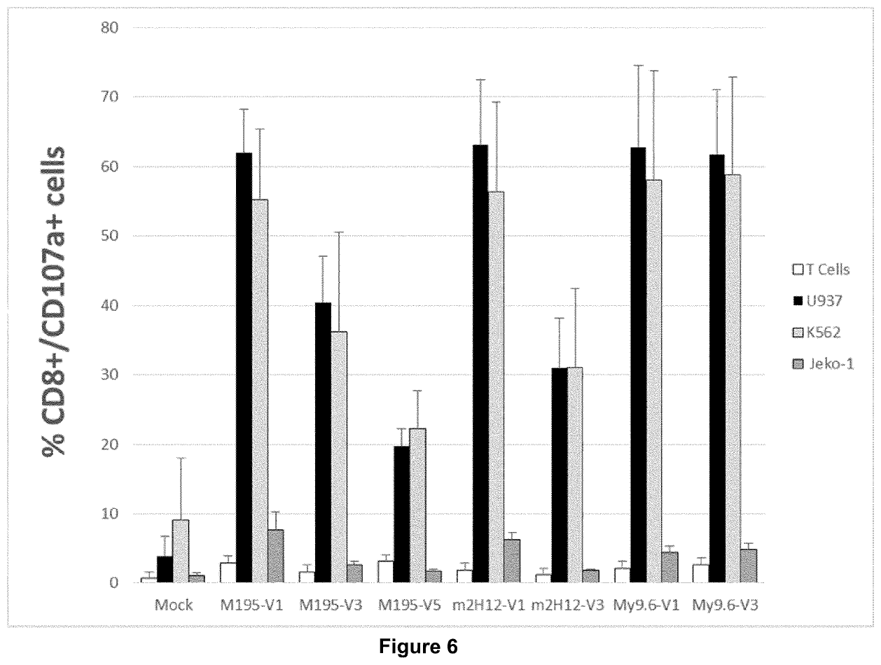

FIG. 6 shows degranulation activity (% CD8/CD107a+ cells) of 7 constructions: M195-V1, M195-V3, M195-V5, m2H12-V1, m2H12-V3 and My9.6-V1 and My9.6-V3 (v1: FcgRIII-hinge/CD8-transmembrane, v3: CD8-hinge/CD8-transmembrane, v5: IgG1-hinge/CD8-transmembrane), when CAR+ T-cells were co-cultured for 6 hours with cells expressing high or intermediate levels of CD33 (U937 and K562, respectively), or with cells that do not express CD33 (Jeko-1). The data is presented as a percentage (%) of degranulation and mean fluorescence activity (MFI).

Interferon Gamma Release and Cell Lysis Induced by T Cells Expressing CD33 CAR Generated Using Anti-CD33 scFv Antibody Fragments Derived from M195, M2H12, and My9.6

For this, T-cells from various donors were isolated from buffy-coat samples and activated using CD3/CD28 beads. Cells were transiently transfected with mRNAs encoding the different candidates at D11 after activation.

CAR activity was assessed by measuring the IFN gamma release, and the cytotoxic activity when co-cultured with cells expressing various levels of CD33 as follows.

### IFN Gamma Release Assay

Anti-CD33 CAR-expressing T-cells were incubated in 96-well plates (40,000 cells/well), together with cell lines expressing various levels of the CD33 protein. Co-cultures were maintained in a final volume of 100 μl of X-Vivo™-15 medium (Lonza) for 24 hours at 37° C. with 5% CO₂. After this incubation period the plates were centrifuged at 1500 rpm for 5 minutes and the supernatants were recovered in a new plate. IFN gamma detection in the cell culture supernatants was done by ELISA assay. The IFN gamma release assays were carried by starting the cell co-cultures 24 h after mRNA transfection.

FIG. 7 shows the amount of IFN gamma released by CD33 car T-cells (T cells) when co-cultured for 24 h with cells expressing different levels of CD33 (U937 and K562), or with cells that do not express CD33 (Jeko-1). IFN gamma release from T-cells cultured alone, in the same conditions that the co-cultures, is also shown. The experiments were done for three independent donors, and results from a representative donor are shown here.

T cells expressing CD33 CAR generated using anti-CD33 scFv antibody fragments derived from M195, M2H12, and My9.6 were then tested for their capacity to lyse CD33+ expressing target cancer cells (U937: Human leukemic monocyte lymphoma cell and K562 human leukemic cell derived from of a patient with chronic myelogenous leukemia (CML) as follows.

### Cytotoxicity Assay

Anti-CD33 CAR-expressing T-cells were incubated in 96-well plates (100,000 cells/well), together with 10,000 target cells (expressing CD33) and 10,000 control (CD33neg) cells in the same well. Target and control cells were labelled with fluorescent intracellular dyes (CFSE or Cell Trace Violet) before co-culturing them with CAR+ T-cells. The co-cultures were incubated for 4 hours at 37° C. with 5% CO₂. After this incubation period, cells were labelled with a fixable viability dye and analyzed by flow cytometry. Viability of each cellular population (target cells or CD33neg control cells) was determined and the % of specific cell lysis was calculated. Cytotoxicity assays were carried out 48 h after mRNA transfection.

FIG. 8 shows the specific cytolytic activity of CAR-T cells. Assays were done 48 h after CAR mRNA transfection. T-cells were co-cultured with U937+Jeko or K562+Jeko cells for 4 hours. Cellular viability for each of the cell lines was determined at the end of the co-cultured and a specific cell lysis percentage was calculated.

All constructions were active, with My9.6 V3 (My9.6-3, SEQ ID NO 68) CAR exhibiting the higher overall activity as compared to control than other CARS.

The above examples demonstrate that the exemplified CAR structures (V1, V3 and V5) comprising a VH-Linker-VL sequence binding to CD33 are active in all assay.

CARs having the structures V1 and V3 were found particularly active in the degranulation assay, INFy release assay and cytotoxic assay see FIGS. 4 to 8.

The present invention provides therefore an anti-CD33 CAR expressing T cell active against cancer cells.

M195-1
(SEQ ID NO. 1 + SEQ ID NO. 19)

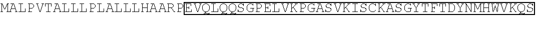

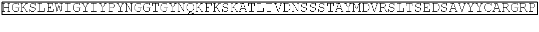

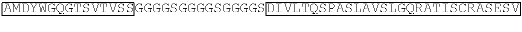

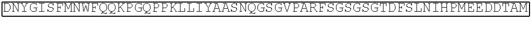

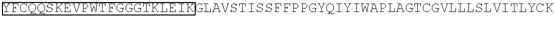

RGRKKLLYIFKQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAEAYSEIGMKG
ERRRGKGHDGLYQGLSTATKDTYDALHMQALPPR
M195-2
(SEQ ID NO. 1 + SEQ ID NO. 20)

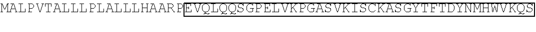

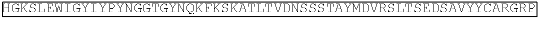

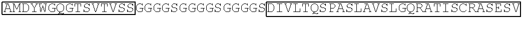

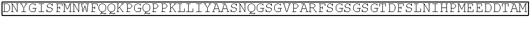

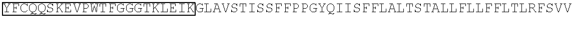

KRGRKKLLYIFKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMAEAYSEIGMK
GERRRGKGHDGLYQGLSTATKDTYDALHMQALPPR
M195-3
(SEQ ID NO. 1 + SEQ ID NO. 21)

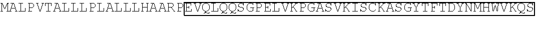

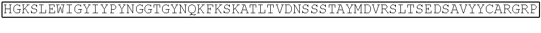

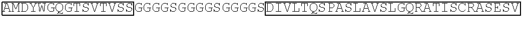

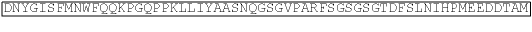

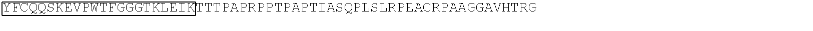

LDFACDIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTYDALHMQALP
PR
M195-4
(SEQ ID NO. 1 + SEQ ID NO. 22)

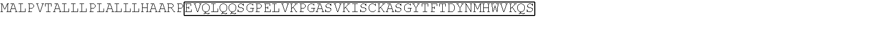

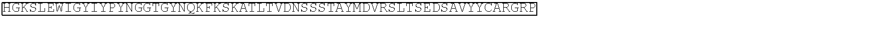

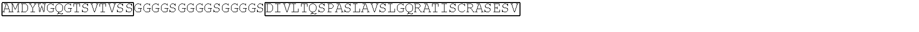

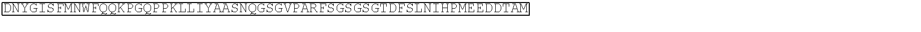

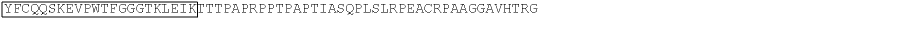

LDFACDIISFFLALTSTALLFLLFFLTLRFSVVKRGRKKLLYIFKQPFMRPVQTTQEEDGCSCR
FPEEEEGGCELRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATKDTYDALHMQ
ALPPR
M195-5
(SEQ ID NO. 1 + SEQ ID NO. 23)

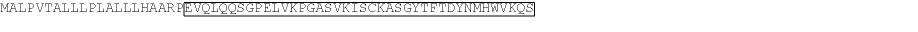

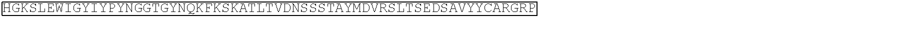

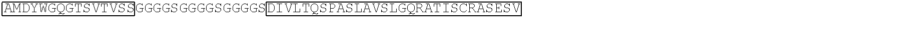

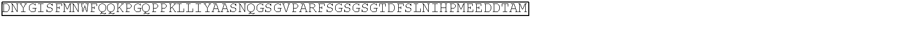

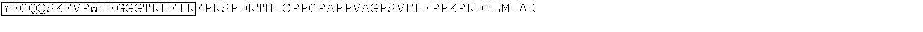

TPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYT
QKSLSLSPGKIYIWAPLAGTCGVLLLSLVITLYCKRGRKKLLYIFKQPFMRPVQTTQEEDGCS
CRFPEEEEGGCELRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTYDALH
MQALPPR
M195-6
(SEQ ID NO. 1 + SEQ ID NO. 24)

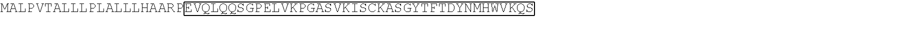

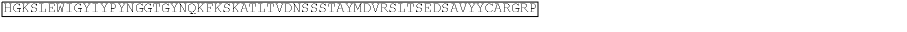

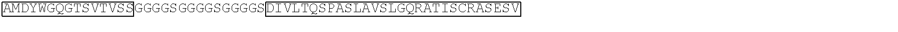

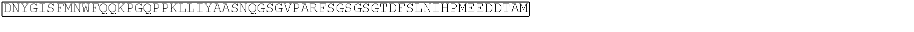

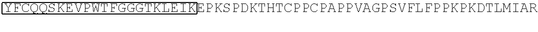

TPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYT
QKSLSLSPGKIISFFLALTSTALLFLLFFLTLRFSVVKRGRKKLLYIFKQPFMRPVQTTQEEDG
CSCRFPEEEEGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDA
LHMQALPPR
m2H12-1
(SEQ ID NO. 1 + SEQ ID NO. 25)

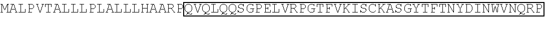

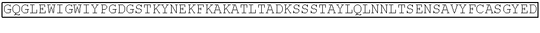

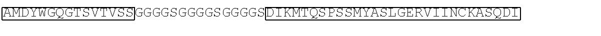

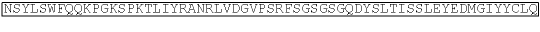

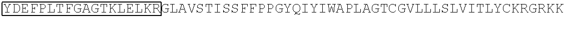

LLYIFKQPFMRPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEIGMKGERRRG
KGHDGLYQGLSTATKDTYDALHMQALPPR
m2H12-2
(SEQ ID NO. 1 + SEQ ID NO. 26)

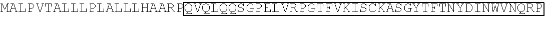

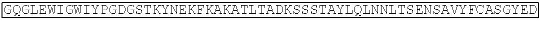

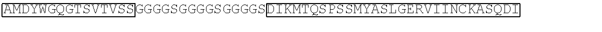

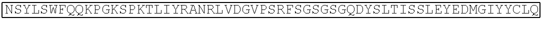

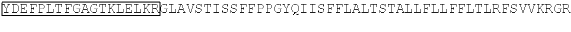

KKLLYIFKQPFMRPVQTTQEEDGCSCRFPEEEEGGCELRVKFSRSADAPAYQQGQNQLYN
ELNLGRREEYDVLDKRRGRDPEMGGKPRRKNPQEGLYNELQKDKMAEAYSEIGMKGERR
RGKGHDGLYQGLSTATKDTYDALHMQALPPR
m2H12-3
(SEQ ID NO. 1 + SEQ ID NO. 27)

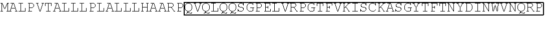

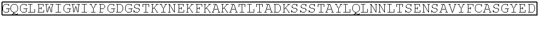

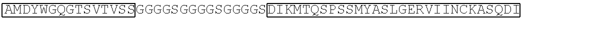

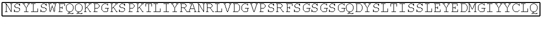

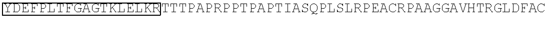

DIYIWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
m2H12-4
(SEQ ID NO. 1 + SEQ ID NO. 28)

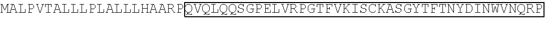

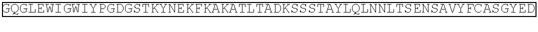

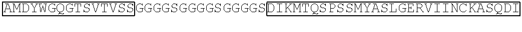

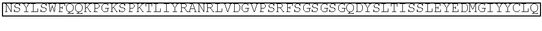

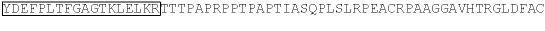

DIISFFLALTSTALLFLLFFLTLRFSVVKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
m2H12-5
(SEQ ID NO. 1 + SEQ ID NO. 29)

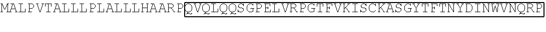

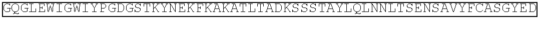

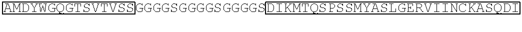

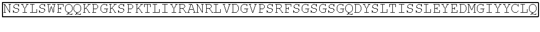

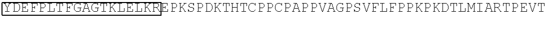

CVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSL
SLSPGKIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTYDALHMQALP
PR
m2H12-6
(SEQ ID NO. 1 + SEQ ID NO. 30)

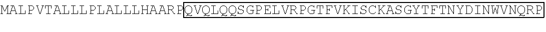

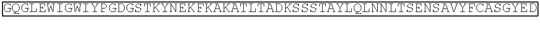

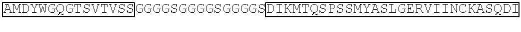

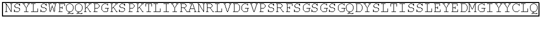

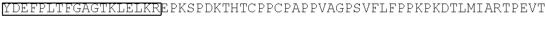

CVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSL
SLSPGKIISFFLALTSTALLFLLFFLTLRFSVVKRGRKKLLYIFKQPFMRPVQTTQEEDGCSCR
FPEEEEGGCELRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATKDTYDALHMQ
ALPPR
DRB2-1
(SEQ ID NO. 1 + SEQ ID NO. 31)

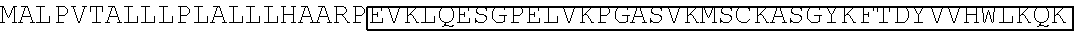

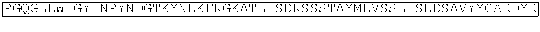

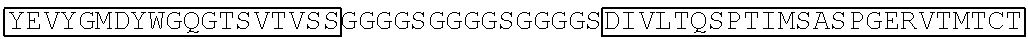

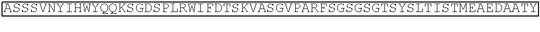

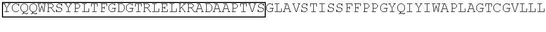

SLVITLYCKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
DRB2-2
(SEQ ID NO. 1 + SEQ ID NO. 32)

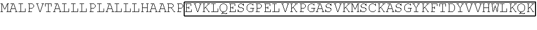

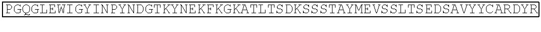

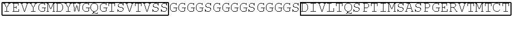

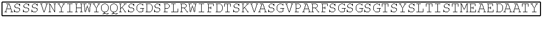

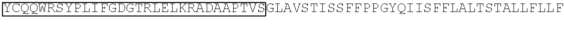

FLTLRFSVVKRGRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
DRB2-3
(SEQ ID NO. 1 + SEQ ID NO. 33)

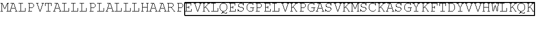

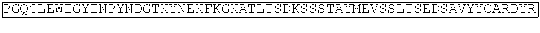

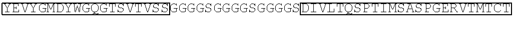

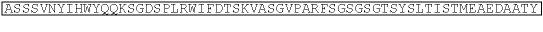

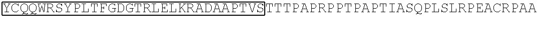

GGAVHTRGLDFACDIYIWAPLAGTCGVLLLSLVITLYCKRGRKKLLYIFKQPFMRPVQTTQEE
DGCSCRFPEEEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKDTY
DALHMQALPPR
DRB2-4
(SEQ ID NO. 1 + SEQ ID NO. 34)

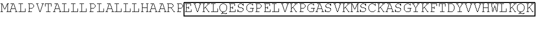

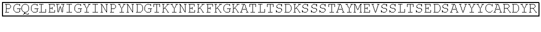

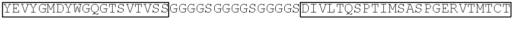

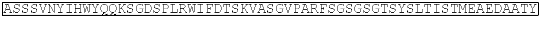

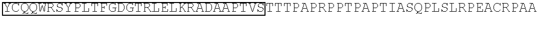

GGAVHTRGLDFACDIISFFLALTSTALLFLLFFLTLRFSVVKRGRKKLLYIFKQPFMRPVQTTQ
EEDGCSCRFPEEEEGGCELRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKD
TYDALHMQALPPR
DRB2-5
(SEQ ID NO. 1 + SEQ ID NO. 35)

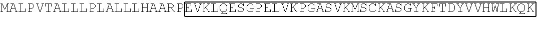

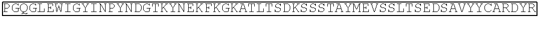

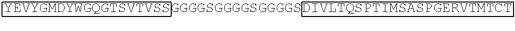

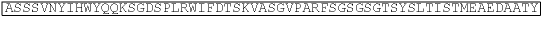

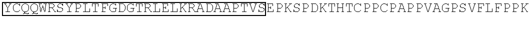

PKDTLMIARTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMH
EALHNHYTQKSLSLSPGKIYIWAPLAGTCGVLLLSLVITLYCKRGRKKLLYIFKQPFMRPVQTT
QEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATK
DTYDALHMALPPR
DRB2-6
(SEQ ID NO. 1 + SEQ ID NO. 36)

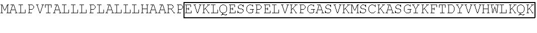

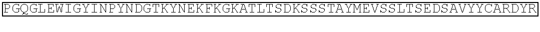

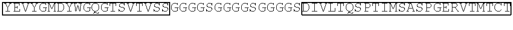

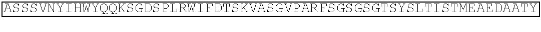

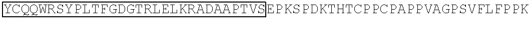

PKDTLMIARTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMH
EALHNHYTQKSLSLSPGKIISFFLALTSTALLFLLFFLTLRFSVVKRGRKKLLYIFKQPFMRPVQ
TTQEEDGCSCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTA
TKDTYDALHMQALPPR
My9.6-1
(SEQ ID NO. 1 + SEQ ID NO. 37)

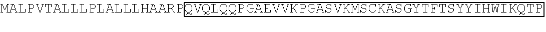

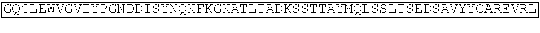

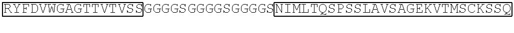

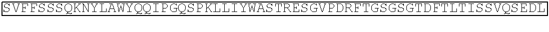

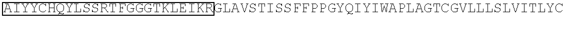

KRGRKKLLYIFKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMAEAYSEIGMK
GERRRGKGHDGLYQGLSTATKDTYDALHMQALPPR
My9.6-2
(SEQ ID NO. 1 + SEQ ID NO. 38)

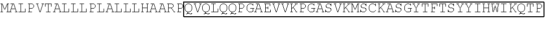

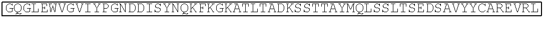

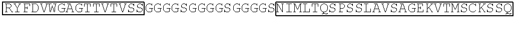

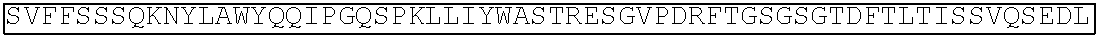

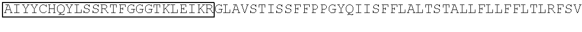

VKRGRKKLLYIFKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDKMAEAYSEIGM
KGERRRGKGHDGLYQGLSTATKDTYDALHMQALPPR
My9.6-3
(SEQ ID NO. 1 + SEQ ID NO. 39)

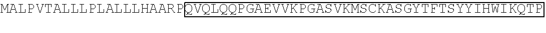

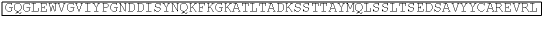

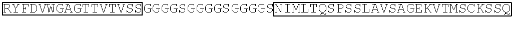

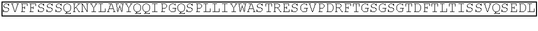

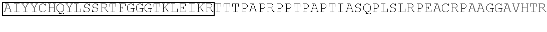

GLDFACDIYIWAPLAGTCGVLLLSLVITLYCKRGRKKLLYIFKQPFMRPVQTTQEEDGCSCRF
PEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQA
LPPR
My9.6-4
(SEQ ID NO. 1 + SEQ ID NO. 40)

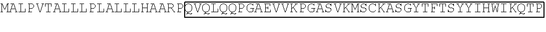

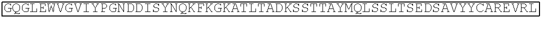

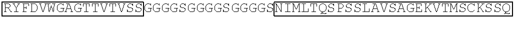

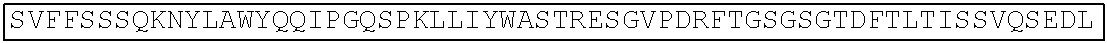

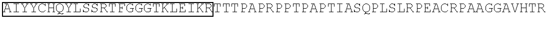

GLDFACDIISFFLALTSTALLFLLFFLTLRFSVVKRGRKKLLYIFKQPFMRPVQTTQEEDGCSC
RFPEEEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHM
QALPPR
My9.6-5
(SEQ ID NO. 1 + SEQ ID NO. 41)

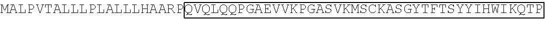

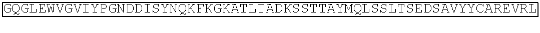

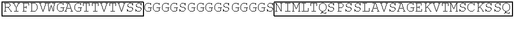

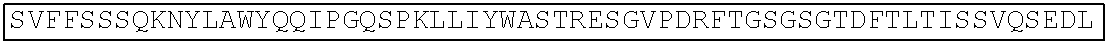

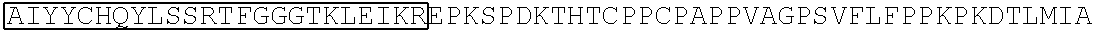

RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHY
TQKSLSLSPGKIYIWAPLAGTCGVLLLSLVITLYCKRGRKKLLYIFKQPFMRPVQTTQEEDGC
SCRFPEEEEGGCELRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDTYDAL
HMQALPPR
My9.6-6
(SEQ ID NO. 1 + SEQ ID NO. 42)

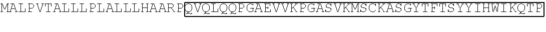

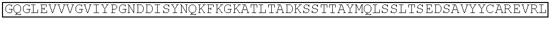

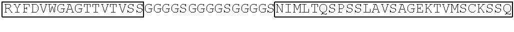

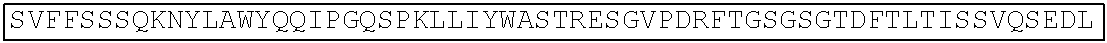

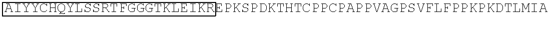

RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHY
TQKSLSLSPGKIISFFLALTSTALLFLLFFLTLRFSVVKRGRKKLLYIFKQPFMRPVQTTQEED
GCSCRFPEEEEGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQGLSTATKDTYD
ALHMQALPPR

### REFERENCES

Arimondo, P. B., C. J. Thomas, et al. (2006). “Exploring the cellular activity of camptothecin-triple-helix-forming oligonucleotide conjugates.” Mol Cell Biol 26(1): 324-33.
Atkins, J. F., N. M. Wills, et al. (2007). “A case for “StopGo”: reprogramming translation to augment codon meaning of GGN by promoting unconventional termination (Stop) after addition of glycine and then allowing continued translation (Go).” Rna 13(6): 803-10.
Bierer, B. E., G. Hollander, et al. (1993). “Cyclosporin A and FK506: molecular mechanisms of immunosuppression and probes for transplantation biology.” Curr Opin Immunol 5(5): 763-73.
Boch, J., H. Scholze, et al. (2009). “Breaking the code of DNA binding specificity of TAL-type III effectors.” Science 326(5959): 1509-12.
Choulika, A., A. Perrin, et al. (1995). “Induction of homologous recombination in mammalian chromosomes by using the I-SceI system of Saccharomyces cerevisiae.” Mol Cell Biol 15(4): 1968-73.
Christian, M., T. Cermak, et al. (2010). “Targeting DNA double-strand breaks with TAL effector nucleases.” Genetics 186(2): 757-61.
Cong, L., F. A. Ran, et al. (2013). “Multiplex genome engineering using CRISPR/Cas systems.” Science 339(6121): 819-23.
Cros, E. et al. (2004). “Problems related to resistance to cytarabine in acute myeloid leukemia”. Leukemia & Lymphoma. 45(6):1123-1132.
Deltcheva, E., K. Chylinski, et al. (2011). “CRISPR RNA maturation by trans-encoded small RNA and host factor RNase III.” Nature 471(7340): 602-7.
Donnelly, M. and G. Elliott (2001). “Nuclear localization and shuttling of herpes simplex virus tegument protein VP13/14.” J Virol 75(6): 2566-74.
Doronina, V. A., C. Wu, et al. (2008). “Site-specific release of nascent chains from ribosomes at a sense codon.” Mol Cell Biol 28(13): 4227-39.
Eisenschmidt, K., T. Lanio, et al. (2005). “Developing a programmed restriction endonuclease for highly specific DNA cleavage.” Nucleic Acids Res 33(22): 7039-47.
Gardin, C. et al. (2007). “Postremission treatment of elderly patients with acute myeloid leukemia in first complete remission after intensive induction chemotherapy: results of the multicenter randomized Acute Leukemia French Association (ALFA) 9803 trial”. Blood. 109(12):5129-5135.
Garneau, J. E., M. E. Dupuis, et al. (2010). “The CRISPR/Cas bacterial immune system cleaves bacteriophage and plasmid DNA.” Nature 468(7320): 67-71.
Gasiunas, G., R. Barrangou, et al. (2012). “Cas9-crRNA ribonucleoprotein complex mediates specific DNA cleavage for adaptive immunity in bacteria.” Proc Natl Acad Sci USA 109(39): E2579-86.
Henderson, D. J., I. Naya, et al. (1991). “Comparison of the effects of FK-506, cyclosporin A and rapamycin on IL-2 production.” Immunology 73(3): 316-21.
Jena, B., G. Dotti, et al. (2010). “Redirecting T-cell specificity by introducing a tumor-specific chimeric antigen receptor.” Blood 116(7): 1035-44.
Jinek, M., K. Chylinski, et al. (2012). “A programmable dual-RNA-guided DNA endonuclease in adaptive bacterial immunity.” Science 337(6096): 816-21.
June, C. H. et al. (2011). “T Cells with Chimeric Antigen Receptors Have Potent Antitumor Effects and Can Establish Memory in Patients with Advanced Leukemia”. Sci. Transl. Med. 3(95):ra73.
Kalish, J. M. and P. M. Glazer (2005). “Targeted genome modification via triple helix formation.” Ann N Y Acad Sci 1058: 151-61.
Li, T., S. Huang, et al. (2011). “TAL nucleases (TALNs): hybrid proteins composed of TAL effectors and FokI DNA-cleavage domain.” Nucleic Acids Res 39(1): 359-72.
Liu, J., M. W. Albers, et al. (1992). “Inhibition of T cell signaling by immunophilin-ligand complexes correlates with loss of calcineurin phosphatase activity.” Biochemistry 31(16): 3896-901.
Mali, P., L. Yang, et al. (2013). “RNA-guided human genome engineering via Cas9.” Science 339(6121): 823-6.
Maniecki, M. B. et al., (2011) “Is hepatotoxicity in patients treated with gemtuzumabozogamicin due to specific targeting of hepatocytes ?”. Leukemia Research e84-e86.
Moscou, M. J. and A. J. Bogdanove (2009). “A simple cipher governs DNA recognition by TAL effectors.” Science 326(5959): 1501.
Paques, F. and P. Duchateau (2007). “Meganucleases and DNA double-strand break-induced recombination: perspectives for gene therapy.” Curr Gene Ther 7(1): 49-66.
Park, T. S., S. A. Rosenberg, et al. (2011). “Treating cancer with genetically engineered T cells.” Trends Biotechnol 29(11): 550-7.
Peipp, M., D. Saul, et al. (2004). “Efficient eukaryotic expression of fluorescent scFv fusion proteins directed against CD antigens for FACS applications.” J Immunol Methods 285(2): 265-80.
Perrin, A., M. Buckle, et al. (1993). “Asymmetrical recognition and activity of the I-SceI endonuclease on its site and on intron-exon junctions.” Embo J 12(7): 2939-47.
Pingoud, A. and G. H. Silva (2007). “Precision genome surgery.” Nat Biotechnol 25(7): 743-4.
Porteus, M. H. and D. Carroll (2005). “Gene targeting using zinc finger nucleases.” Nat Biotechnol 23(8): 967-73.
Rouet, P., F. Smih, et al. (1994). “Introduction of double-strand breaks into the genome of mouse cells by expression of a rare-cutting endonuclease.” Mol Cell Biol 14(12): 8096-106.
Schwemmlein. M. et al. (2006). “A CD33-specific single-chain immunotoxin mediates potent apoptosis of cultured human myeloid leukaemia cells”. British Journal of Haematology. 133(2): 141-151
Sorek, R., C. M. Lawrence, et al. (2013). “CRISPR-mediated Adaptive Immune Systems in Bacteria and Archaea.” Annu Rev Biochem.
Stoddard, B. L. (2005). “Homing endonuclease structure and function.” Q Rev Biophys 38(1): 49-95.
Vitale, C. et al. (2001). “Surface expression and function of p75/AIRM-1 or CD33 in acute myeloid leukemias: engagement of CD33 induces apoptosis of leukemic cells”. PNAS 98:5764-5769.

1-42. (canceled)

43. A method of engineering an immune cell for the treatment of a hematological cancer expressing CD33 comprising introducing a polynucleotide encoding an anti-CD33 chimeric antigen receptor (CAR) comprising an extracellular ligand binding domain comprising a heavy chain variable region (V_{H}) and a light chain variable region (V_{L}) from a monoclonal anti-CD33 antibody into the immune cell to produce an anti-CD33 CAR engineered immune cell.

44. The method of claim 43, wherein the method further comprises activating and/or expanding the anti-CD33 CAR engineered immune cell.

45. The method of claim 44, wherein the activation and/or expansion comprises exposing the anti-CD33 CAR engineered immune cell to a calcium ionophore, phorbol 12-myristate 13-acetate (PMA), a mitogenic lectin, or a combination thereof.

46. The method of claim 44, wherein the activation and/or expansion comprises exposing the anti-CD33 CAR engineered immune cell to an anti-CD3 antibody, an anti-CD2 antibody, an anti-CD28 antibody, a protein kinase C activator, or a combination thereof.

47. The method of claim 44, wherein a gene encoding CD33 has been inactivated in the anti-CD33 CAR engineered immune cell.

48. The method of claim 44, wherein a gene encoding one or more component of T-cell receptor (TCR) has been inactivated in the anti-CD33 CAR engineered immune cell.

49. The method of claim 44, wherein a gene encoding or regulating HLA or β2m protein expression has been inactivated in the anti-CD33 CAR engineered immune cell.

50. The method of claim 44, wherein the anti-CD33 CAR engineered immune cell is resistant to at least one of an immunosuppressive drug or a chemotherapy drug.

51. The method of claim 44, wherein the polynucleotide encoding the anti-CD33 CAR is comprised in a vector.

52. The method of claim 51, wherein the vector is a lentiviral vector.

53. The method of claim 44, wherein the anti-CD33 CAR comprises a CD8α hinge and a CD8α transmembrane domain.

54. The method of claim 53, wherein the CD8α hinge comprises an amino acid sequence set forth in SEQ ID NO: 4, and the CD8α transmembrane domain comprises an amino acid sequence set forth in SEQ ID NO: 6.

55. The method of claim 44, wherein the anti-CD33 CAR comprises a FcγRIIIa hinge and a CD8α transmembrane domain.

56. The method of claim 55, wherein the FcγRIIIa hinge comprises an amino acid sequence set forth in SEQ ID NO: 3, and the CD8α transmembrane domain comprises an amino acid sequence set forth in SEQ ID NO: 6.

57. The method of claim 44, wherein the anti-CD33 CAR comprises an IgG1 hinge and a CD8α transmembrane domain.

58. The method of claim 57, wherein the IgG1 hinge comprises an amino acid sequence set forth in SEQ ID NO: 5, and the CD8α transmembrane domain comprises an amino acid sequence set forth in SEQ ID NO: 6.

59. The method of claim 44, wherein the anti-CD33 CAR comprises a CD3-ζ signaling domain having an amino acid sequence set forth in SEQ ID NO: 9, and a co-stimulatory domain from 4-1BB having an amino acid sequence set forth in SEQ ID NO: 8.

60. The method of claim 44, wherein:
the V_{H} region comprises SEQ ID NO: 11 and the V_{L} region comprises SEQ ID NO: 12;
the V_{H} region comprises SEQ ID NO: 13 and the V_{L} region comprises SEQ ID NO: 14;
the V_{H} region comprises SEQ ID NO: 15 and the V_{L} region comprises SEQ ID NO: 16; or
the V_{H} region comprises SEQ ID NO: 17 and the V_{L} region comprises SEQ ID NO: 18.

61. The method of claim 44, wherein the anti-CD33 CAR comprises SEQ ID NO: 19, SEQ ID NO: 25, SEQ ID NO: 31, SEQ ID NO: 37, SEQ ID NO: 21, SEQ ID NO: 27, SEQ ID NO: 33, SEQ ID NO: 39, SEQ ID NO: 23, SEQ ID NO: 29, SEQ ID NO: 35, or SEQ ID NO: 41.

62. The method of claim 44, wherein the engineered immune cell is an inflammatory T-lymphocyte, a cytotoxic T-lymphocyte, a regulatory T-lymphocyte, or a helper T-lymphocyte.


SEQUENCE LISTING (87 sequences)

SEQ ID NO: 1 - protein, 20 aa, synthetic construct
  REGION 1..20: note = description of artificial sequence: synthetic oligopeptide
  REGION 1..20: note = signal peptide
METDTLLLWVLLLWVPGSTG

SEQ ID NO: 2 - protein, 21 aa, synthetic construct
  REGION 1..21: note = description of artificial sequence: synthetic oligopeptide
  REGION 1..21: note = signal peptide
MALPVTALLLPLALLLHAARP

SEQ ID NO: 3 - protein, 16 aa, Homo sapiens
  REGION 1..16: note = FcgRIIIa hinge
GLAVSTISSFFPPGYQ

SEQ ID NO: 4 - protein, 45 aa, Homo sapiens
  REGION 1..45: note = CD8a hinge
TTTPAPRPPTPAPTIASQPLSLRPEACRPAAGGAVHTRGLDFACD

SEQ ID NO: 5 - protein, 231 aa, Homo sapiens
  REGION 1..231: note = IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, Homo sapiens
  REGION 1..24: note = CD8alpha transmembrane domain
IYIWAPLAGTCGVLLLSLVITLYC

SEQ ID NO: 7 - protein, 27 aa, Homo sapiens
  REGION 1..27: note = 41BB transmembrane domain
IISFFLALTSTALLFLLFFLTLRFSVV

SEQ ID NO: 8 - protein, 42 aa, Homo sapiens
  REGION 1..42: note = Fragment of 4-1BB (residues 214-255)
KRGRKKLLYIFKQPFMRPVQTTQEEDGCSCRFPEEEEGGCEL

SEQ ID NO: 9 - protein, 112 aa, Homo sapiens
  REGION 1..112: note = fragment of T-cell surface glycoprotein CD3 zeta chain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, synthetic construct
  REGION 1..15: note = description of artificial sequence: synthetic oligopeptide
  REGION 1..15: note = linker
GGGGSGGGGSGGGGS

SEQ ID NO: 11 - protein, 115 aa, synthetic construct
  REGION 1..115: note = description of artificial sequence: synthetic oligopeptide
  REGION 1..115: note = M195 heavy chain variable region
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVS

SEQ ID NO: 12 - protein, 111 aa, synthetic construct
  REGION 1..111: note = description of artificial sequence: synthetic oligopeptide
  REGION 1..111: note = M195 light chain variable region
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 13 - protein, 117 aa, synthetic construct
  REGION 1..117: note = description of artificial sequence: synthetic oligopeptide
  REGION 1..117: note = m2H12 heavy chain variable region
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSS

SEQ ID NO: 14 - protein, 108 aa, synthetic construct
  REGION 1..108: note = description of artificial sequence: synthetic oligopeptide
  REGION 1..108: note = m2H12 light chain variable region
DIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKR

SEQ ID NO: 15 - protein, 120 aa, synthetic construct
  REGION 1..120: note = description of artificial sequence: synthetic oligopeptide
  REGION 1..120: note = DRB2 heavy chain variable region
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSS

SEQ ID NO: 16 - protein, 115 aa, synthetic construct
  REGION 1..115: note = description of artificial sequence: synthetic oligopeptide
  REGION 1..115: note = DRB2 light chain variable region
DIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVS

SEQ ID NO: 17 - protein, 118 aa, synthetic construct
  REGION 1..118: note = description of artificial sequence: synthetic oligopeptide
  REGION 1..118: note = My9-6 heavy chain variable region
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 18 - protein, 113 aa, synthetic construct
  REGION 1..113: note = description of artificial sequence: synthetic oligopeptide
  REGION 1..113: note = My9-6 light chain variable region
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 19 - protein, 436 aa, synthetic construct
  REGION 1..436: note = description of artificial sequence: synthetic oligopeptide
  REGION 1..436: note = M195-1 polypeptide CAR sequence
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 439 aa, synthetic construct
  REGION 1..439: note = description of artificial sequence: synthetic oligopeptide
  REGION 1..439: note = M195-2 polypeptide CAR sequence
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 465 aa, synthetic construct
  REGION 1..465: note = description of artificial sequence: synthetic oligopeptide
  REGION 1..465: note = M195-3 polypeptide CAR sequence
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 468 aa, synthetic construct
  REGION 1..468: note = description of artificial sequence: synthetic oligopeptide
  REGION 1..468: note = M195-4 polypeptide CAR sequence
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 651 aa, synthetic construct
  REGION 1..651: note = description of artificial sequence: synthetic oligopeptide
  REGION 1..651: note = M195-5 polypeptide CAR sequence
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 654 aa, synthetic construct
  REGION 1..654: note = description of artificial sequence: synthetic oligopeptide
  REGION 1..654: note = M195-6 polypeptide CAR sequence
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 434 aa, synthetic construct
  REGION 1..434: note = description of artificial sequence: synthetic oligopeptide
  REGION 1..434: note = m2H12-1 polypeptide CAR sequence
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 437 aa, synthetic construct
  REGION 1..437: note = description of artificial sequence: synthetic oligopeptide
  REGION 1..437: note = m2H12-2 polypeptide CAR sequence
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 463 aa, synthetic construct
  REGION 1..463: note = description of artificial sequence: synthetic oligopeptide
  REGION 1..463: note = m2H12-3 polypeptide CAR sequence
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 466 aa, synthetic construct
  REGION 1..466: note = description of artificial sequence: synthetic oligopeptide
  REGION 1..466: note = m2H12-4 polypeptide CAR sequence
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 649 aa, synthetic construct
  REGION 1..649: note = description of artificial sequence: synthetic oligopeptide
  REGION 1..649: note = m2H12-5 polypeptide CAR sequence
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 652 aa, synthetic construct
  REGION 1..652: note = description of artificial sequence: synthetic oligopeptide
  REGION 1..652: note = m2H12-6 polypeptide CAR sequence
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 444 aa, synthetic construct
  REGION 1..444: note = description of artificial sequence: synthetic oligopeptide
  REGION 1..444: note = DRB2-1 polypeptide CAR sequence
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 447 aa, synthetic construct
  REGION 1..447: note = description of artificial sequence: synthetic oligopeptide
  REGION 1..447: note = DRB2-2 polypeptide CAR sequence
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 473 aa, synthetic construct
  REGION 1..473: note = description of artificial sequence: synthetic oligopeptide
  REGION 1..473: note = DRB2-3 polypeptide CAR sequence
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 476 aa, synthetic construct
  REGION 1..476: note = description of artificial sequence: synthetic oligopeptide
  REGION 1..476: note = DRB2-4 polypeptide CAR sequence
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 659 aa, synthetic construct
  REGION 1..659: note = description of artificial sequence: synthetic oligopeptide
  REGION 1..659: note = DRB2-5 polypeptide CAR sequence
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 662 aa, synthetic construct
  REGION 1..662: note = description of artificial sequence: synthetic oligopeptide
  REGION 1..662: note = DRB2-6 polypeptide CAR sequence
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 440 aa, synthetic construct
  REGION 1..440: note = description of artificial sequence: synthetic oligopeptide
  REGION 1..440: note = My9-6-1 polypeptide CAR sequence
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 443 aa, synthetic construct
  REGION 1..443: note = description of artificial sequence: synthetic oligopeptide
  REGION 1..443: note = My9-6-2 polypeptide CAR sequence
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 469 aa, synthetic construct
  REGION 1..469: note = description of artificial sequence: synthetic oligopeptide
  REGION 1..469: note = My9-6-3 polypeptide CAR sequence
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 472 aa, synthetic construct
  REGION 1..472: note = description of artificial sequence: synthetic oligopeptide
  REGION 1..472: note = My9-6-4 polypeptide CAR sequence
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 655 aa, synthetic construct
  REGION 1..655: note = description of artificial sequence: synthetic oligopeptide
  REGION 1..655: note = My9-6-5 polypeptide CAR sequence
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 658 aa, synthetic construct
  REGION 1..658: note = description of artificial sequence: synthetic oligopeptide
  REGION 1..658: note = My9-6-6 polypeptide CAR sequence
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 49 aa, synthetic construct
  REGION 1..49: note = description of artificial sequence: synthetic oligopeptide
  REGION 1..49: note = Target TALEN TRAC_T01
TTGTCCCACAGATATCCAGAACCCTGACCCTGCCGTGTACCAGCTGAGA

SEQ ID NO: 44 - protein, 530 aa, synthetic construct
  REGION 1..530: note = description of artificial sequence: synthetic oligopeptide
  REGION 1..530: note = TAL binding domainTRAC_T01-L
LTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALE

SEQ ID NO: 45 - protein, 530 aa, synthetic construct
  REGION 1..530: note = description of artificial sequence: synthetic oligopeptide
  REGION 1..530: note = TAL binding domain TRAC_T01-R
LTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALE

SEQ ID NO: 46 - DNA, 2814 bp, synthetic construct
  misc_feature 1..2814: note = description of artificial sequence: synthetic polynucleotide
  misc_feature 1..2814: note = polynucleotide encodingTRAC_T01-L TALEN
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 47 - DNA, 2832 bp, synthetic construct
  misc_feature 1..2832: note = description of artificial sequence: synthetic polynucleotide
  misc_feature 1..2832: note = polynucleotide encoding TRAC_T01-R TALEN
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 48 - protein, 457 aa, synthetic construct
  REGION 1..457: note = M195-1
MALPVTALLLPLALLLHAARPEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 460 aa, synthetic construct
  REGION 1..460: note = M195-2
MALPVTALLLPLALLLHAARPEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 486 aa, synthetic construct
  REGION 1..486: note = M195-3
MALPVTALLLPLALLLHAARPEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 489 aa, synthetic construct
  REGION 1..489: note = M195-4
MALPVTALLLPLALLLHAARPEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 672 aa, synthetic construct
  REGION 1..672: note = M195-5
MALPVTALLLPLALLLHAARPEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 675 aa, synthetic construct
  REGION 1..675: note = M195-6
MALPVTALLLPLALLLHAARPEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 455 aa, synthetic construct
  REGION 1..455: note = m2H12-1
MALPVTALLLPLALLLHAARPQVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 458 aa, synthetic construct
  REGION 1..458: note = m2H12-2
MALPVTALLLPLALLLHAARPQVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 484 aa, synthetic construct
  REGION 1..484: note = m2H12-3
MALPVTALLLPLALLLHAARPQVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 487 aa, synthetic construct
  REGION 1..487: note = m2H12-4
MALPVTALLLPLALLLHAARPQVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 670 aa, synthetic construct
  REGION 1..670: note = m2H12-5
MALPVTALLLPLALLLHAARPQVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 673 aa, synthetic construct
  REGION 1..673: note = m2H12-6
MALPVTALLLPLALLLHAARPQVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 465 aa, synthetic construct
  REGION 1..465: note = DRB2-1
MALPVTALLLPLALLLHAARPEVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 468 aa, synthetic construct
  REGION 1..468: note = DRB2-2
MALPVTALLLPLALLLHAARPEVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 494 aa, synthetic construct
  REGION 1..494: note = DRB2-3
MALPVTALLLPLALLLHAARPEVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 497 aa, synthetic construct
  REGION 1..497: note = DRB2-4
MALPVTALLLPLALLLHAARPEVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 680 aa, synthetic construct
  REGION 1..680: note = DRB2-5
MALPVTALLLPLALLLHAARPEVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 683 aa, synthetic construct
  REGION 1..683: note = DRB2-6
MALPVTALLLPLALLLHAARPEVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 461 aa, synthetic construct
  REGION 1..461: note = My9.6-1
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 464 aa, synthetic construct
  REGION 1..464: note = My9.6-2
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGLAVSTISSFFPPGYQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 490 aa, synthetic construct
  REGION 1..490: note = My9.6-3
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 493 aa, synthetic construct
  REGION 1..493: note = My9.6-4
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRTTTPAPRPPTPAPTIASQPLSLRPEACRPAAGGAVHTRGLDFACDIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 676 aa, synthetic construct
  REGION 1..676: note = My9.6-5
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 679 aa, synthetic construct
  REGION 1..679: note = My9.6-6
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - DNA, 44 bp, synthetic construct
  misc_feature 1..44: note = TALEN1_CD33_Exon3
  source 1..44: mol_type = other DNA
tgcatcccctctttctcctcactagacttgacccacaggcccaa

SEQ ID NO: 73 - DNA, 49 bp, synthetic construct
  misc_feature 1..49: note = TALEN2_CD33_Exon3
  source 1..49: mol_type = other DNA
ttctcctcactagacttgacccacaggcccaaaatcctcatccctggca

SEQ ID NO: 74 - DNA, 49 bp, synthetic construct
  misc_feature 1..49: note = TALEN1_CD33_Exon4
  source 1..49: mol_type = other DNA
tcctctcctagatgttccacagaacccaacaactggtatctttccagga

SEQ ID NO: 75 - DNA, 44 bp, synthetic construct
  misc_feature 1..44: note = TALEN2_CD33_Exon4
  source 1..44: mol_type = other DNA
tcctagatgttccacagaacccaacaactggtatctttccagga

SEQ ID NO: 76 - DNA, 17 bp, synthetic construct
  misc_feature 1..17: note = Sequence bound by TALEN1 Left exon 3
  source 1..17: mol_type = other DNA
tgcatcccctctttctc

SEQ ID NO: 77 - DNA, 17 bp, synthetic construct
  misc_feature 1..17: note = Sequence bound by TALEN2 Left exon 3
  source 1..17: mol_type = other DNA
ttctcctcactagactt

SEQ ID NO: 78 - DNA, 17 bp, synthetic construct
  misc_feature 1..17: note = Sequence bound by TALEN1 Left exon 4
  source 1..17: mol_type = other DNA
tcctctcctagatgttc

SEQ ID NO: 79 - DNA, 17 bp, synthetic construct
  misc_feature 1..17: note = Sequence bound by TALEN2 Left exon 4
  source 1..17: mol_type = other DNA
tcctagatgttccacag

SEQ ID NO: 80 - DNA, 10 bp, synthetic construct
  misc_feature 1..10: note = spacer 1 (Exon3)
  source 1..10: mol_type = other DNA
ctcactagac

SEQ ID NO: 81 - DNA, 15 bp, synthetic construct
  misc_feature 1..15: note = spacer2 (Exon3)
  source 1..15: mol_type = other DNA
gacccacaggcccaa

SEQ ID NO: 82 - DNA, 15 bp, synthetic construct
  misc_feature 1..15: note = spacer 1 (Exon4)
  source 1..15: mol_type = other DNA
cacagaacccaacaa

SEQ ID NO: 83 - DNA, 10 bp, synthetic construct
  misc_feature 1..10: note = spacer 2 (Exon4)
  source 1..10: mol_type = other DNA
aacccaacaa

SEQ ID NO: 84 - DNA, 17 bp, synthetic construct
  misc_feature 1..17: note = Sequence bound by TALEN1 right exon 3
  source 1..17: mol_type = other DNA
ttgacccacaggcccaa

SEQ ID NO: 85 - DNA, 17 bp, synthetic construct
  misc_feature 1..17: note = Sequence bound by TALEN2 right exon 3
  source 1..17: mol_type = other DNA
aatcctcatccctggca

SEQ ID NO: 86 - DNA, 17 bp, synthetic construct
  misc_feature 1..17: note = Sequence bound by TALEN1 right exon 4
  source 1..17: mol_type = other DNA
ctggtatctttccagga

SEQ ID NO: 87 - DNA, 17 bp, synthetic construct
  misc_feature 1..17: note = Sequence bound by TALEN2 right exon 4
  source 1..17: mol_type = other DNA
ctggtatctttccagga